(19) United States
(12) Patent Application Publication
(10) Pub. No.: US 2023/0227912 A1
(43) Pub. Date: Jul. 20, 2023

(54) METHODS AND BIOMARKERS FOR DIAGNOSTICS, DISEASE MONITORING, PERSONALIZED DRUG DISCOVERY AND TARGETED THERAPY OF MALIGNANT AND NEURODEGENERATIVE DISEASE CONDITIONS

(71) Applicant: BAR-ILAN UNIVERSITY, Ramat Gan (IL)

(72) Inventors: Milana FRENKEL-MORGENSTERN, Gilon (IL); Rajesh DETROJA, Safed (IL); Vikrant PALANDE, Safed (IL)

(73) Assignee: BAR-ILAN UNIVERSITY, Ramat Gan (IL)

(21) Appl. No.: 17/777,467

(22) PCT Filed: Nov. 17, 2020
(86) PCT No.: PCT/IL2020/051188

§ 371 Date: May 17, 2022

Related U.S. Application Data
(60) Provisional application No. 62/936,537, filed on Nov. 17, 2019.

(51) Int. Cl. C12Q 1/6886; C12Q 1/6883; G16B 20/00

(52) U.S. Cl. CPC C12Q 1/6886; C12Q 1/6883; G16B 20/00

(57) ABSTRACT

Compositions, methods and biomarkers for diagnostics, monitoring and therapy of various health and complex-disease conditions, such as malignant and neurodegenerative disorders, utilizing the techniques of data mining, computational biology, artificial intelligence and molecular biology are provided.

This application is the U.S. national phase of PCT Application No. PCT/IL2020/051188 filed Nov. 17, 2020, which claims the benefit of U.S. provisional application Ser. No. 62/936,537 filed Nov. 17, 2019, the disclosures of which are hereby incorporated in their entirety by reference herein.

### REFERENCE TO AN ELECTRONIC SEQUENCE LISTING

The contents of the electronic sequence listing (PCTIL2020051188-SQL Corrected_MIL-P-002-US_ST25.txt; Size: 96,635 bytes; and Date of Creation: Apr. 4, 2023) is herein incorporated by reference in its entirety. No new matter is added by this incorporation.

### FIELD OF THE INVENTION

The present invention relates to compositions, methods and biomarkers for diagnostics, monitoring and therapy of various health and complex malignant and/or neurodegenerative disease conditions, utilizing the techniques of data mining, computational biology, artificial intelligence and molecular biology.

### BACKGROUND OF THE INVENTION

Aberrations such as chromosomal translocations, trans-splicing, fusions and gene variants are frequently found in human disorders. Chromosomal translocations may result in a chimeric gene expressing a fusion transcript which is then translated into a fusion protein that affects normal regulatory pathways. Cancer is one of the most prominent examples of such disorder. Liquid biopsy is a newly emerging technique for cancer diagnostics and being widely accepted because of its non-invasive approach and many advantages over biopsy-based diagnostics [1]. This technique uses circulating cell-free nucleic acid fragment, namely circulating cell-free DNA (cfDNA) fragments and/or circulating free RNA (cfRNA). CfDNA is free floating small fragments of nucleic acids/DNA in the blood plasma that are not associated with cells or cell fragments. CfDNA has been shown to be present in patients with various types of neoplasms and is not thought to be directly related to metastasis. This cfDNA may be analyzed for specific genetic markers of neoplasm with varying degrees of specificity and sensitivity. CfDNA that are released into the blood stream by the tumor tissue can be screened for the cancer specific mutations for diagnostics of cancers. Unlike biopsy-based diagnosis, liquid biopsy can be repeated multiple times, which gives a profile of real time mutations in tumor and, more importantly, represents tumor heterogeneity [2]. One of the main challenges in liquid biopsy is to detect rare mutation in cfDNA which is accompanied with large amount of wild type cfDNA fragments. Rate of mutation varies among different cancer types and cancer grades and, thus, its availability in blood cfDNA is directly correlated. These mutations can be missed during cfDNA isolation step or at the detection level, where it may not get sequenced on next generation sequencing platform or amplified on droplet PCR due to its rare availability. These detection errors can then lead to false negative diagnostics [3]. Matthew W., et. al. in 2016 showed that it is possible to identify tissues contributing to cell-free DNA by looking at their fragmentation patterns, instead of looking for specific mutations in the DNA [4]. Recent studies suggested that cfDNA analysis may be useful for diagnostics for additional health and complex-disease conditions, such as neurodegenerative disorders.

Current methods and systems for analyzing the genetic markers of humans and providing tailor-made therapeutic means are still very limited. Therefore, there is an urgent, unmet need in game-changing technologies based on data mining and computational biology which will enable generating a characterization of the condition and generating a therapy model configured to correct the condition, thus providing tailored and personalized treatment solutions.

### SUMMARY OF THE INVENTION

The invention provides a novel powerful method for identifying, monitoring and treating a condition in a subject, wherein said condition is associated with omics-discoverable features.

In one embodiment, the invention provides method for identifying a condition in a subject, wherein said condition is characterized by omics-discoverable features; the method comprising:
a. obtaining a biological sample from the at least one subject, wherein said biological sample comprises cell-free nucleic acids;
b. sequencing said cell-free nucleic acids;
c. mapping the sequencing results to the reference human genome;
d. identifying unmapped non-linear reads;
e. mapping the unmapped non-linear reads to the pre-computed sequence data set indicative of said condition; and
a. identifying at least one sequence associated with said condition.

The invention further provides a method for treating a condition in a subject, wherein said condition is characterized by omics-discoverable features, the method comprising:
a. obtaining a biological sample from at least one subject, wherein said biological sample comprises cell-free nucleic acids;
b. sequencing said cell-free nucleic acids;
c. mapping the sequencing results to the reference human genome;
d. identifying unmapped non-linear reads;
e. mapping the unmapped non-linear reads to the pre-computed sequence data set indicative of said condition;
f. identifying at least one sequence associated with said condition;
g. applying a pre-computed treatment model to identify therapeutic means suitable for treating the condition;
h. identifying the therapeutic means for treating the condition based on the pre-computed treatment model; and
i. providing the human subject with the therapeutic means to thereby effectively treat the condition in the subject.

The invention further provides therapeutic means for use in the treatment of a neurodegenerative disorder characterized by omics-discoverable features in a subject, wherein said therapeutic means are identified by applying a pre-computed treatment model designed to identify the therapeutic means suitable for treating said neurodegenerative disorder; and, wherein said identifying of the therapeutic means comprises the steps of:
a. obtaining a biological sample from at least one subject wherein said biological sample comprises cell-free nucleic acids;
b. sequencing said cell-free nucleic acids;
c. mapping the sequencing results to the reference human genome;
d. identifying unmapped non-linear reads;
e. mapping of pre-computed sequence data set with the unmapped non-linear reads;
f. identifying sequences associated with said condition; and,
g. applying a pre-computed treatment model to identify therapeutic means suitable for treating the condition.

The invention further provides therapeutic means for use in the treatment of a malignant disorder characterized by omics-discoverable features in a subject, wherein said therapeutic means are identified by applying a pre-computed treatment model designed to identify the therapeutic means suitable for treating said malignant disorder; and, wherein said identifying of the therapeutic means comprises the steps of:
a. obtaining a biological sample from at least one subject wherein said biological sample comprises cell-free nucleic acids;
b. sequencing said cell-free nucleic acids;
c. mapping the sequencing results to the reference human genome;
d. identifying unmapped non-linear reads;
e. mapping the unmapped non-linear reads to the pre-computed sequence data set indicative of said condition;
f. identifying sequences associated with said condition; and,
g. applying a pre-computed treatment model to identify therapeutic means suitable for treating the condition.

The invention further provides an isolated nucleotide sequence having at least 75% sequence identity to a nucleotide sequence selected from the group consisting of SEQ ID NO:1-32.

The invention further provides an isolated nucleotide sequence having at least 75% sequence identity to a nucleotide sequence selected from the group consisting of SEQ ID NO:33-64.

The invention yet further provides an isolated nucleotide sequence having at least 75% sequence identity to a nucleotide sequence selected from the group consisting of SEQ ID NO:65-77.

The invention yet further provides an isolated nucleotide sequence having at least 75% sequence identity to a nucleotide sequence selected from the group consisting of SEQ ID NO:78-85.

The invention yet further provides an isolated nucleotide sequence having at least 75% sequence identity to a nucleotide sequence selected from the group consisting of SEQ ID NO: 86-114.

The invention yet further provides an isolated nucleotide sequence selected from the group consisting of nucleotide sequences set forth as SEQ ID NO:1-114.

### BRIEF DESCRIPTION OF THE DRAWINGS

Exemplary non-limited embodiments of the disclosed subject matter will be described, with reference to the following description of the embodiments, in conjunction with the figures. The figures are generally not shown to scale and any sizes are only meant to be exemplary and not necessarily limiting, corresponding or like elements are optionally designated by the same numerals or letters.

FIG. 1 is a graphic illustration of an exemplary embodiment of method used for quantitative and qualitative analysis of cfDNA in glioblastoma patients: Blood collected from both healthy and glioma patients (before surgery) and centrifuged to separate plasma. As shown in figure, silica membrane spin-column based technique is used to separate DNA, and fragment size and concentration of cfDNA was estimated by Bioanalyzer and Qubit;

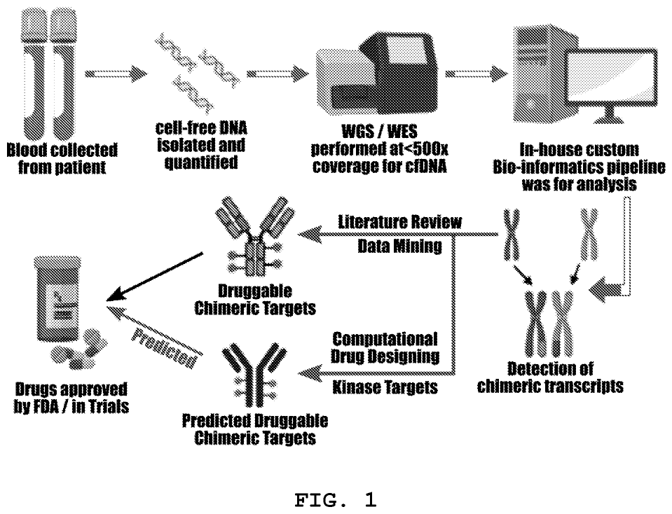

FIG. 2 shows electophoregrams showing 6 types of DNA fragment enrichments;

FIG. 3 demonstrates DNA fragment sizes in healthy volunteers and glioblastoma patients;

FIG. 4 is a graphic illustration of an exemplary embodiment of method for mutation analysis of cfDNA and ctDNA of glioblastoma patients: Blood and tumor biopsy samples are obtained from five GBMs patients, to obtain cfDNA, gDNA, and tomour DNA. Both cfDNA and ctDNA were tested for the presence of mutations in top-50 selected genes from COSMIC database that are commonly mutated in GBMs.

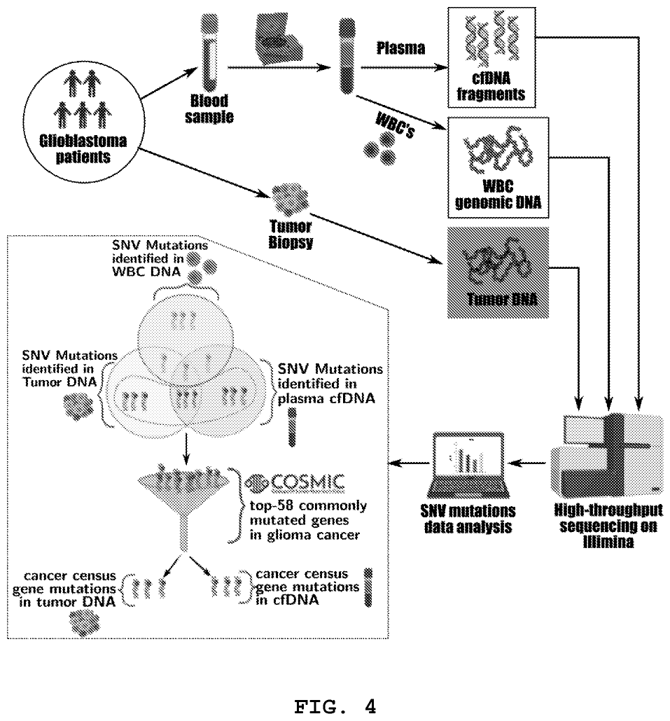

### DETAILED DESCRIPTION OF THE INVENTION

The present invention is now described more fully hereinafter. This invention may, however, be embodied in many different forms and should not be construed as limited to the embodiments set forth herein; rather these embodiments are provided so that this disclosure will be thorough and complete and will fully convey the scope of the invention to those skilled in the art.

In one embodiment of the invention, provided a method for identifying a condition in a subject, wherein said condition is characterized by omics-discoverable features; the method comprising:
a. obtaining a biological sample from the at least one subject, wherein said biological sample comprises cell-free nucleic acids;
b. sequencing said cell-free nucleic acids;
c. mapping the sequencing results to the reference human genome;
d. identifying unmapped non-linear reads;
e. mapping the unmapped non-linear reads to the pre-computed sequence data set indicative of said condition; and
f. identifying at least one sequence associated with said condition.

In one embodiment, the method of the invention further includes a step of isolating circulating cell-free nucleic acids from the biological sample. In the context of the invention, the phrase “a method of identifying a condition” is meant to be understood, without limitation, as diagnostic means, namely a method for diagnosing, recognizing, detecting and monitoring various characteristics of a certain disease or a condition.

As used herein, the phrase “pre-computed sequence data” refers, without limitation, to a pre-computed set of nucleotide sequences found in databases or generated using some heuristic or combination as random sequences; or merged as parts of sequences to a set of novel sequences, including separated and merged exons, introns, genes, pseudogenes or any genomic sequences and/or chimeric RNA sequences or fusion genes sequences that cannot be mapped to human genome linearly; and genomic “integrations” of pathogens to human genome.

As used herein the term “omics” refers, without limitation to genomics, proteomics, metagenomics, methylomics, epigenomics, and metabolomics. As used herein the term “biological sample” refers, without limitation to any biological material collected from a subject. A non-limiting list of biological samples of the invention includes blood, serum, plasma, urine, saliva, amniotic fluid, feces, synovial fluid, peritoneal fluid, pleural fluid, lymphatic fluid, mucus, and cerebrospinal fluid (CSF), or any other body fluid or acceptable body tissue. In one embodiment, the biological sample is a liquid biological sample. In another embodiment, the biological sample is selected from the group consisting of blood, serum, plasma, urine, saliva and cerebrospinal fluid (CSF). As used herein, the term “circulating cell-free nucleic acids” refers, without limitation to degraded nucleic acid fragments released to the blood plasma or other body fluids. In one embodiment, the circulating cell-free nucleic acids are selected from the group consisting of circulating cell-free RNA, circulating cell-free DNA, circulating cell free nucleic acid complexes, and circulating cell-free microRNA. In another embodiment, the circulating cell-free nucleic acids is circulating cell-free DNA. As used herein, the term “sequence” refers, without limitation to oligonucleotide or polynucleotide. As used herein, the terms “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene”, “allele” or “gene sequence” is used broadly to refer to a DNA (deoxynucleic nucleic acids) associated with a biological function. Thus, genes may include introns and exons as in the genomic sequence or may comprise only a coding sequence as in cDNAs, and/or may include cDNAs in combination with regulatory sequences. Thus, according to the various aspects of the invention, genomic DNA, cDNA or coding DNA may be used.

As used herein, the term “neurodegenerative disorder” refers, without limitation to a range of conditions which primarily affect the neurons in the human brain and tend to worsen over time. In one embodiment, the neurodegenerative disorder is selected from the group consisting of Alzheimer's disease, dementia, and Parkinson's disease. In one embodiment the neurodegenerative disorder is Alzheimer's disease.

As used herein, the term “malignant disorder” refers, without limitation to a condition in which abnormal cells divide without control and can invade nearby tissues. Malignant cells can also spread to other parts of the body through the blood and lymph systems. In the context of the invention, “malignant disorder” can be replaced by any of the following terms: cancer, neoplasm, tumor or any other acceptable term that relates to pathological conditions accompanied by abnormal cell growth. A non-limiting list of malignant disorders of the invention includes sarcoma, carcinoma, melanoma, glioma, glioblastoma, lymphoma, astrocytoma, Grade I—pilocytic astrocytoma, Grade II—Low-grade astrocytoma, Grade III—anaplastic astrocytoma, chordoma, CNS lymphoma, craniopharyngioma, brain stem glioma, ependymoma, medulloblastoma, and meningioma and any other tumor type.

According to some embodiments, the sequence associated with said condition is a gene fusion. The term “gene fusion” refers to a chimeric genomic DNA resulting from the fusion of at least a portion of a first gene to a portion of a second gene. The point of transition between the sequences from the first gene in the fusion to the sequences from the second gene in the fusion is referred to as the “breakpoint” or “fusion point” and/or “chimeric junction site”. Transcription of the gene fusion results in a chimeric mRNA and/or chimeric RNA transcript. As used herein in, the term “chimeric RNA transcript” refers, without limitation, to single-stranded sequences of RNAs transcribed from various locations in the total genome corresponding to exons and/or introns from two different genes or non-linear combination of exons/introns of the same gene; two copies of the same gene; regions of pathogen genome, which fuse together to produce a single RNA transcript and/or a single cell free DNA molecule. Two unrelated genomic loci on different chromosomes may produce a chimeric transcript through a genomic rearrangement event or due to trans-splicing. Similarly, a read-through transcript of two adjacent genomic loci may produce chimeric RNAs. As used herein, the term “gene” refers, without limitation, to a polynucleotide (e.g., a DNA segment), that encodes a polypeptide and includes regions preceding and following the coding regions as well as intervening sequences (introns) between individual coding segments (exons).

According to some embodiments, the at least one sequence associated with the condition of the invention is selected from the group consisting of sequences having at least 75% sequence identity to the nucleotide sequence set forth as SEQ ID NO:1-32. In one embodiment, the sequence associated with said condition is selected from the group consisting of sequences having 75%-98% sequence identity to the nucleotide sequence set forth as SEQ ID NO: 1-32. In another embodiment, the sequence associated with said condition is selected from the group consisting of sequences having at least 80%-98%, 85%-98%, 87%-98%, 90%-98%, and 95%-98% sequence identity to the nucleotide sequence set forth as SEQ ID NO: 1-32. In one embodiment, the sequence associated with said condition is selected from the group consisting of sequences having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99% sequence identity to the nucleotide sequence set forth as SEQ ID NO: 1-32. In another embodiment, the at least one sequence associated with the condition of the invention is selected from the group consisting of sequences set forth as SEQ ID NO: 1-32.

According to some embodiments, the sequence associated with the condition of the invention is selected from the group consisting of sequences having at least 75% sequence identity to the nucleotide sequence set forth as SEQ ID NO: 33-64. In one embodiment, the sequence associated with said condition is selected from the group consisting of sequences having at least 75%-98% sequence identity to the nucleotide sequence set forth as SEQ ID NO: 33-64. In another embodiment, the sequence associated with said condition is selected from the group consisting of sequences having at least 80%-98%, 85%-98%, 87%-98%, 90%-98%, and 95%-98% sequence identity to the nucleotide sequence set forth as SEQ ID NO: 33-64. In one embodiment, the sequence associated with said condition is selected from the group consisting of sequences having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99% sequence identity to the nucleotide sequence set forth as SEQ ID NO: 33-64. In one embodiment, the at least one sequence associated with the condition of the invention is selected from the group consisting of nucleotide sequences set forth as SEQ ID NO: 33-64.

According to some embodiments, the sequence associated with the condition of the invention is selected from the group consisting of sequences having at least 75% sequence identity to the nucleotide sequence set forth as SEQ ID NO: 65-77. In one embodiment, the sequence associated with said condition is selected from the group consisting of sequences having at least 75%-98% sequence identity to the nucleotide sequence set forth as SEQ ID NO: 65-77. In another embodiment, the sequence associated with said condition is selected from the group consisting of sequences having at least 80%-98%, 85%-98%, 87%-98%, 90%-98%, and 95%-98% sequence identity to the nucleotide sequence set forth as SEQ ID NO: 65-77. In one embodiment, the sequence associated with said condition is selected from the group consisting of sequences having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99% sequence identity to the nucleotide sequence set forth as SEQ ID NO: 65-77. In one embodiment, the at least one sequence associated with the condition of the invention is selected from the group consisting of nucleotide sequences set forth as SEQ ID NO: 65-77.

According to some embodiments, the sequence associated with the condition of the invention is selected from the group consisting of sequences having at least 75% sequence identity to the nucleotide sequence set forth as SEQ ID NO: 78-85. In one embodiment, the sequence associated with said condition is selected from the group consisting of sequences having at least 75%-98% sequence identity to the nucleotide sequence set forth as SEQ ID NO: 78-85. In another embodiment, the sequence associated with said condition is selected from the group consisting of sequences having at least 80%-98%, 85%-98%, 87%-98%, 90%-98%, and 95%-98% sequence identity to the nucleotide sequence set forth as SEQ ID NO: 78-85. In one embodiment, the sequence associated with said condition is selected from the group consisting of sequences having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99% sequence identity to the nucleotide sequence set forth as SEQ ID NO: 78-85. In one embodiment, the at least one sequence associated with the condition of the invention is selected from the group consisting of nucleotide sequences set forth as SEQ ID NO: 78-85.

According to some embodiments, the sequence associated with the condition of the invention is selected from the group consisting of sequences having at least 75% sequence identity to the nucleotide sequence set forth as SEQ ID NO: 86-114. In one embodiment, the sequence associated with said condition is selected from the group consisting of sequences having at least 75%-98% sequence identity to the nucleotide sequence set forth as SEQ ID NO: 86-114. In another embodiment, the sequence associated with said condition is selected from the group consisting of sequences having at least 80%-98%, 85%-98%, 87%-98%, 90%-98%, and 95%-98% sequence identity to the nucleotide sequence set forth as SEQ ID NO: 86-114. In one embodiment, the sequence associated with said condition is selected from the group consisting of sequences having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99% sequence identity to the nucleotide sequence set forth as SEQ ID NO: 86-114. In one embodiment, the at least one sequence associated with the condition of the invention is selected from the group consisting of nucleotide sequences set forth as SEQ ID NO: 86-114.

As used herein, “sequence identity” or “identity” in the context of two nucleic acid sequences makes reference to the residues in the two sequences that are the same when aligned for maximum correspondence over a specified comparison window. The term further refers hereinafter to the amount of characters which match exactly between two different sequences. Hereby, gaps are not counted, and the measurement is relational to the shorter of the two sequences. It is further within the scope that the terms “similarity” and “identity” additionally refer to local homology, identifying domains that are homologous or similar (in nucleotide sequence). It is acknowledged that bioinformatics tools such as BLAST, SSEARCH, FASTA, and HMMER calculate local sequence alignments which identify the most similar region between two sequences. For domains that are found in different sequence contexts in different proteins, the alignment should be limited to the homologous domain, since the domain homology is providing the sequence similarity captured in the score. According to some aspects the term similarity or identity further includes a sequence motif, which is a nucleotide or amino-acid sequence pattern that is widespread and has, or is conjectured to have, a biological significance.

In the context of the invention, the phrase “omics-discoverable features” is meant to be understood as a characteristic that can be identified and/or recognized and/or measured by means of “omics” as defined above. In one embodiment, the omics-discoverable feature is selected from the group consisting of genomics-discoverable features, proteomics-discoverable features, metagenomics-discoverable features, methylomics-discoverable features, epigenomics-discoverable features, and metabolomics-discoverable features. The non-limiting list of omics-discoverable features of the invention include chimeras, chimeric RNAS, gene-gene fusions, sense-antisense (SAS) chimeras, genomic integrations, aberrations, inversions, and other genomic alterations.

According to some embodiments, the invention provides a method for treating a condition in a subject, wherein said condition is characterized by omics-discoverable features, the method comprising:
a. obtaining a biological sample from at least one subject, wherein said biological sample comprises cell-free nucleic acids;
b. sequencing said cell-free nucleic acids;
c. mapping the sequencing results to the reference human genome;
d. identifying unmapped non-linear reads;
e. mapping the unmapped non-linear reads to the pre-computed sequence data set indicative of said condition;
f. identifying at least one sequence associated with said condition;
g. applying a pre-computed treatment model to identify therapeutic means suitable for treating the condition;
h. identifying the therapeutic means for treating the condition based on the pre-computed treatment model; and
i. providing the subject with the therapeutic means to thereby effectively treat the condition in the human subject.

In one embodiment, the method further comprises the step of isolating circulating cell-free nucleic acids from the biological sample. As described herein, isolation of cell-free nucleic acids may be done by any suitable technique known in the art, commercially available or using in-house developed tools and proprietary technology. In one embodiment, the biological sample is selected from the group consisting of blood, serum, plasma, urine, saliva, amniotic fluid, feces, synovial fluid, peritoneal fluid, tissue biopsy, pleural fluid, lymphatic fluid, mucus, and cerebrospinal fluid (CSF). In another embodiment, the biological sample is a liquid biological sample. In one embodiment, the circulating cell-free nucleic acids is selected from the group consisting of circulating cell-free RNA, circulating cell-free DNA, circulating cell free nucleic acid complexes, and circulating cell-free microRNA.

According to some embodiments, the condition associated with omics-discoverable features is a neurodegenerative disorder. In one embodiment, the neurodegenerative disorder is selected from the group consisting of Alzheimer's disease (AD), dementia, Parkinson's disease (PD), PD-related disorders, Prion disease, Motor neuron diseases (MND), Huntington's disease (HD), Spinocerebellar ataxia (SCA), and Spinal muscular atrophy (SMA).

According to some embodiments, the condition associated with omics-discoverable features is a malignant disorder. In one embodiment, the malignant disorder is selected from the group consisting of sarcoma, carcinoma, melanoma, glioma, glioblastoma, lymphoma, astrocytoma, Grade I—pilocytic astrocytoma, Grade II—Low-grade astrocytoma, Grade III—anaplastic astrocytoma, chordoma, CNS lymphoma, craniopharyngioma, brain stem glioma, ependymoma, medulloblastoma, and meningioma and other tumor types.

According to some embodiments, in the method of the invention, the at least one sequence associated with the condition of the invention is selected from the group consisting of sequences having at least 75% sequence identity to the nucleotide sequence set forth as SEQ ID NO:1-32. In one embodiment, the sequence associated with said condition is selected from the group consisting of sequences having 75%-98% sequence identity to the nucleotide sequence set forth as SEQ ID NO: 1-32. In another embodiment, the sequence associated with said condition is selected from the group consisting of sequences having at least 80%-98%, 85%-98%, 87%-98%, 90%-98%, and 95%-98% sequence identity to the nucleotide sequence set forth as SEQ ID NO: 1-32. In one embodiment, the sequence associated with said condition is selected from the group consisting of sequences having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99% sequence identity to the nucleotide sequence set forth as SEQ ID NO: 1-32. In another embodiment, the at least one sequence associated with the condition of the invention is selected from the group consisting of sequences set forth as SEQ ID NO: 1-32.

According to some embodiments, in the method of the invention, the sequence associated with the condition of the invention is selected from the group consisting of sequences having at least 75% sequence identity to the nucleotide sequence set forth as SEQ ID NO: 33-64. In one embodiment, the sequence associated with said condition is selected from the group consisting of sequences having at least 75%-98% sequence identity to the nucleotide sequence set forth as SEQ ID NO: 33-64. In another embodiment, the sequence associated with said condition is selected from the group consisting of sequences having at least 80%-98%, 85%-98%, 87%-98%, 90%-98%, and 95%-98% sequence identity to the nucleotide sequence set forth as SEQ ID NO: 33-64. In one embodiment, the sequence associated with said condition is selected from the group consisting of sequences having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99% sequence identity to the nucleotide sequence set forth as SEQ ID NO: 33-64. In one embodiment, the at least one sequence associated with the condition of the invention is selected from the group consisting of nucleotide sequences set forth as SEQ ID NO: 33-64.

According to some embodiments, in the method of the invention, the sequence associated with the condition of the invention is selected from the group consisting of sequences having at least 75% sequence identity to the nucleotide sequence set forth as SEQ ID NO: 65-77. In one embodiment, the sequence associated with said condition is selected from the group consisting of sequences having at least 75%-98% sequence identity to the nucleotide sequence set forth as SEQ ID NO: 65-77. In another embodiment, the sequence associated with said condition is selected from the group consisting of sequences having at least 80%-98%, 85%-98%, 87%-98%, 90%-98%, and 95%-98% sequence identity to the nucleotide sequence set forth as SEQ ID NO: 65-77. In one embodiment, the sequence associated with said condition is selected from the group consisting of sequences having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99% sequence identity to the nucleotide sequence set forth as SEQ ID NO: 65-77. In one embodiment, the at least one sequence associated with the condition of the invention is selected from the group consisting of nucleotide sequences set forth as SEQ ID NO: 65-77.

According to some embodiments, in the method of the invention, the sequence associated with the condition of the invention is selected from the group consisting of sequences having at least 75% sequence identity to the nucleotide sequence set forth as SEQ ID NO: 78-85. In one embodiment, the sequence associated with said condition is selected from the group consisting of sequences having at least 75%-98% sequence identity to the nucleotide sequence set forth as SEQ ID NO: 78-85. In another embodiment, the sequence associated with said condition is selected from the group consisting of sequences having at least 80%-98%, 85%-98%, 87%-98%, 90%-98%, and 95%-98% sequence identity to the nucleotide sequence set forth as SEQ ID NO: 78-85. In one embodiment, the sequence associated with said condition is selected from the group consisting of sequences having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99% sequence identity to the nucleotide sequence set forth as SEQ ID NO: 78-85. In one embodiment, the at least one sequence associated with the condition of the invention is selected from the group consisting of nucleotide sequences set forth as SEQ ID NO: 78-85.

According to some embodiments, in the method of the invention, the sequence associated with the condition of the invention is selected from the group consisting of sequences having at least 75% sequence identity to the nucleotide sequence set forth as SEQ ID NO: 86-114. In one embodiment, the sequence associated with said condition is selected from the group consisting of sequences having at least 75%-98% sequence identity to the nucleotide sequence set forth as SEQ ID NO: 86-114. In another embodiment, the sequence associated with said condition is selected from the group consisting of sequences having at least 80%-98%, 85%-98%, 87%-98%, 90%-98%, and 95%-98% sequence identity to the nucleotide sequence set forth as SEQ ID NO: 86-114. In one embodiment, the sequence associated with said condition is selected from the group consisting of sequences having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99% sequence identity to the nucleotide sequence set forth as SEQ ID NO: 86-114. In one embodiment, the at least one sequence associated with the condition of the invention is selected from the group consisting of nucleotide sequences set forth as SEQ ID NO: 86-114.

According to some embodiments, in the method of the invention, omics-discoverable features are selected from the group consisting of genomics-discoverable features, proteomics-discoverable features, metagenomics-discoverable features, methylomics-discoverable features, epigenomics-discoverable features, and metabolomics-discoverable features. In another embodiment, omics-discoverable features are selected from chimeras, chimeric RNAS, gene-gene fusions, sense-antisense (SAS) chimeras, Genomic integrations, aberrations, inversions, and other genomic alterations.

According to some embodiments, in the method of the invention, the therapeutic means are selected from the group consisting of an investigational drug, an approved drug, a food supplement, phototherapy, radiation therapy, surgical intervention, non-invasive image-guided procedure, multi-step treatment protocol, immune-therapy, biological treatment, or any combination thereof.

According to some embodiments, in the method of the invention, the subject is a human subject.

According to some embodiments, in the method of the invention, the subject is a non-human subject.

According to some embodiments the invention provides therapeutic means for use in the treatment of a malignant disorder characterized by omics-discoverable features in a subject, wherein said therapeutic means are identified by applying a pre-computed treatment model designed to identify the therapeutic means suitable for treating said autoimmune disease; and, wherein said identifying of the therapeutic means comprises the steps of:
a. obtaining a biological sample from at least one subject wherein said biological sample comprises cell-free nucleic acids;
b. sequencing said cell-free nucleic acids;
c. mapping the sequencing results to the reference human genome;
d. identifying unmapped non-linear reads;
e. mapping the unmapped non-linear reads to the pre-computed sequence data set indicative of said condition;
f. identifying sequences associated with said condition; and,
g. applying a pre-computed treatment model to identify therapeutic means suitable for treating the condition.

In one embodiment, identifying of the therapeutic means comprises the step of isolating circulating cell-free nucleic acids from the biological sample.

In one embodiment, therapeutic means are selected from the group consisting of an investigational drug, an approved drug, a food supplement, phototherapy, radiation therapy, surgical intervention, non-invasive image-guided procedure, multi-step treatment protocol, immune-therapy or a combination thereof.

In one embodiment, the malignant disorder is selected from the group consisting of sarcoma, carcinoma, melanoma, glioma, glioblastoma, lymphoma, astrocytoma, Grade I—pilocytic astrocytoma, Grade II—Low-grade astrocytoma, Grade III—anaplastic astrocytoma, chordoma, CNS lymphoma, craniopharyngioma, brain stem glioma, ependymoma, medulloblastoma, and meningioma and any other tumor type.

According to some embodiments the invention provides therapeutic means for use in the treatment of a neurodegenerative disorder characterized by omics-discoverable features in a subject, wherein said therapeutic means are identified by applying a pre-computed treatment model designed to identify the therapeutic means suitable for treating said autoimmune disease; and, wherein said identifying of the therapeutic means comprises the steps of:
a. obtaining a biological sample from at least one subject wherein said biological sample comprises cell-free nucleic acids;
b. sequencing said cell-free nucleic acids;
c. mapping the sequencing results to the reference human genome;
d. identifying unmapped non-linear reads;
e. mapping the unmapped non-linear reads to the pre-computed sequence data set indicative of said condition;
f. identifying sequences associated with said condition; and,
g. applying a pre-computed treatment model to identify therapeutic means suitable for treating the condition.

In one embodiment, identifying of the therapeutic means comprises the step of isolating circulating cell-free nucleic acids from the biological sample. In one embodiment, therapeutic means are selected from the group consisting of an investigational drug, an approved drug, a food supplement, phototherapy, radiation therapy, surgical intervention, non-invasive image-guided procedure, multi-step treatment protocol, or a combination thereof. In one embodiment, the neurodegenerative disorder is selected from the group consisting of Alzheimer's disease (AD), dementia, Parkinson's disease (PD), PD-related disorders, Prion disease, Motor neuron diseases (MND), Huntington's disease (HD), Spinocerebellar ataxia (SCA), and Spinal muscular atrophy (SMA).

In one embodiment, provided therapeutic means for use a medicament.

According to some embodiments, in the therapeutic means of the invention, the at least one sequence associated with the disorder of the invention is selected from the group consisting of sequences having at least 75% sequence identity to the nucleotide sequence set forth as SEQ ID NO:1-32. In one embodiment, the sequence associated with said condition is selected from the group consisting of sequences having 75%-98% sequence identity to the nucleotide sequence set forth as SEQ ID NO: 1-32. In another embodiment, the sequence associated with said condition is selected from the group consisting of sequences having at least 80%-98%, 85%-98%, 87%-98%, 90%-98%, and 95%-98% sequence identity to the nucleotide sequence set forth as SEQ ID NO: 1-32. In one embodiment, the sequence associated with said condition is selected from the group consisting of sequences having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99% sequence identity to the nucleotide sequence set forth as SEQ ID NO: 1-32. In another embodiment, the at least one sequence associated with the condition of the invention is selected from the group consisting of sequences set forth as SEQ ID NO: 1-32.

According to some embodiments, in the therapeutic means of the invention, the at least one sequence associated with the disorder of the invention is selected from the group consisting of sequences having at least 75% sequence identity to the nucleotide sequence set forth as SEQ ID NO: 33-64. In one embodiment, the sequence associated with said condition is selected from the group consisting of sequences having at least 75%-98% sequence identity to the nucleotide sequence set forth as SEQ ID NO: 33-64. In another embodiment, the sequence associated with said condition is selected from the group consisting of sequences having at least 80%-98%, 85%-98%, 87%-98%, 90%-98%, and 95%-98% sequence identity to the nucleotide sequence set forth as SEQ ID NO: 33-64. In one embodiment, the sequence associated with said condition is selected from the group consisting of sequences having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99% sequence identity to the nucleotide sequence set forth as SEQ ID NO: 33-64. In one embodiment, the at least one sequence associated with the condition of the invention is selected from the group consisting of nucleotide sequences set forth as SEQ ID NO: 33-64.

According to some embodiments, in the therapeutic means of the invention, the at least one sequence associated with the disorder of the invention is selected from the group consisting of sequences having at least 75% sequence identity to the nucleotide sequence set forth as SEQ ID NO: 65-77. In one embodiment, the sequence associated with said condition is selected from the group consisting of sequences having at least 75%-98% sequence identity to the nucleotide sequence set forth as SEQ ID NO: 65-77. In another embodiment, the sequence associated with said condition is selected from the group consisting of sequences having at least 80%-98%, 85%-98%, 87%-98%, 90%-98%, and 95%-98% sequence identity to the nucleotide sequence set forth as SEQ ID NO: 65-77. In one embodiment, the sequence associated with said condition is selected from the group consisting of sequences having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99% sequence identity to the nucleotide sequence set forth as SEQ ID NO: 65-77. In one embodiment, the at least one sequence associated with the condition of the invention is selected from the group consisting of nucleotide sequences set forth as SEQ ID NO: 65-77.

According to some embodiments, in the therapeutic means of the invention, the at least one sequence associated with the disorder of the invention is selected from the group consisting of sequences having at least 75% sequence identity to the nucleotide sequence set forth as SEQ ID NO: 78-85. In one embodiment, the sequence associated with said condition is selected from the group consisting of sequences having at least 75%-98% sequence identity to the nucleotide sequence set forth as SEQ ID NO: 78-85. In another embodiment, the sequence associated with said condition is selected from the group consisting of sequences having at least 80%-98%, 85%-98%, 87%-98%, 90%-98%, and 95%-98% sequence identity to the nucleotide sequence set forth as SEQ ID NO: 78-85. In one embodiment, the sequence associated with said condition is selected from the group consisting of sequences having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99% sequence identity to the nucleotide sequence set forth as SEQ ID NO: 78-85. In one embodiment, the at least one sequence associated with the condition of the invention is selected from the group consisting of nucleotide sequences set forth as SEQ ID NO: 78-85.

According to some embodiments, in the therapeutic means of the invention, the at least one sequence associated with the disorder of the invention is selected from the group consisting of sequences having at least 75% sequence identity to the nucleotide sequence set forth as SEQ ID NO: 86-114. In one embodiment, the sequence associated with said condition is selected from the group consisting of sequences having at least 75%-98% sequence identity to the nucleotide sequence set forth as SEQ ID NO: 86-114. In another embodiment, the sequence associated with said condition is selected from the group consisting of sequences having at least 80%-98%, 85%-98%, 87%-98%, 90%-98%, and 95%-98% sequence identity to the nucleotide sequence set forth as SEQ ID NO: 86-114. In one embodiment, the sequence associated with said condition is selected from the group consisting of sequences having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99% sequence identity to the nucleotide sequence set forth as SEQ ID NO: 86-114. In one embodiment, the at least one sequence associated with the condition of the invention is selected from the group consisting of nucleotide sequences set forth as SEQ ID NO: 86-114.

According to some embodiments, in the therapeutic means of the invention, the subject is a human subject.

According to some embodiments, in the therapeutic means of the invention, the subject is a non-human subject.

According to some embodiments, the invention provides an isolated nucleotide sequence having at least 75% sequence identity to a nucleotide sequence selected from the group consisting of SEQ ID NO:1-32. In another embodiment, the invention provides an isolated nucleotide sequence having 75%-98% sequence identity to the nucleotide sequence set forth as SEQ ID NO: 1-32. In another embodiment, the invention provides an isolated nucleotide sequence having 80%-98%, 85%-98%, 87%-98%, 90%-98%, and 95%-98% sequence identity to the nucleotide sequence set forth as SEQ ID NO: 1-32. In another embodiment, the invention provides an isolated nucleotide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99% sequence identity to the nucleotide sequence set forth as SEQ ID NO: 1-32.

According to some embodiments, the invention provides an isolated nucleotide sequence selected from the group consisting of sequences set forth as SEQ ID NO: 1-32.

According to some embodiments, the invention provides an isolated nucleotide sequence having at least 75% sequence identity to a nucleotide sequence selected from the group consisting of SEQ ID NO:33-64. In another embodiment, the invention provides an isolated nucleotide sequence having 750-98% sequence identity to the nucleotide sequence set forth as SEQ ID NO: 33-64. In another embodiment, the invention provides an isolated nucleotide sequence having 80%-98%, 85%-98%, 87%-98%, 90%-98%, and 95%-98% sequence identity to the nucleotide sequence set forth as SEQ ID NO: 33-64. In another embodiment, the invention provides an isolated nucleotide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99% sequence identity to the nucleotide sequence set forth as SEQ ID NO: 33-64.

According to some embodiments, the invention provides an isolated nucleotide sequence selected from the group consisting of sequences set forth as SEQ ID NO: 33-64.

According to some embodiments, the invention provides an isolated nucleotide sequence having at least 75% sequence identity to a nucleotide sequence selected from the group consisting of SEQ ID NO:65-77. In another embodiment, the invention provides an isolated nucleotide sequence having 75%-98% sequence identity to the nucleotide sequence set forth as SEQ ID NO: 65-77. In another embodiment, the invention provides an isolated nucleotide sequence having 80%-98%, 85%-98%, 87%-98%, 90%-98%, and 95%-98% sequence identity to the nucleotide sequence set forth as SEQ ID NO: 65-77. In another embodiment, the invention provides an isolated nucleotide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99% sequence identity to the nucleotide sequence set forth as SEQ ID NO: 65-77.

According to some embodiments, the invention provides an isolated nucleotide sequence selected from the group consisting of sequences set forth as SEQ ID NO: 65-77.

According to some embodiments, the invention provides an isolated nucleotide sequence having at least 75% sequence identity to a nucleotide sequence selected from the group consisting of SEQ ID NO:78-85. In another embodiment, the invention provides an isolated nucleotide sequence having 75%-98% sequence identity to the nucleotide sequence set forth as SEQ ID NO: 78-85. In another embodiment, the invention provides an isolated nucleotide sequence having 80%-98%, 85%-98%, 87%-98%, 90%-98%, and 95%-98% sequence identity to the nucleotide sequence set forth as SEQ ID NO: 78-85. In another embodiment, the invention provides an isolated nucleotide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99% sequence identity to the nucleotide sequence set forth as SEQ ID NO: 78-85.

According to some embodiments, the invention provides an isolated nucleotide sequence selected from the group consisting of sequences set forth as SEQ ID NO: 78-85.

According to some embodiments, the invention provides an isolated nucleotide sequence having at least 75% sequence identity to a nucleotide sequence selected from the group consisting of SEQ ID NO:86-114. In another embodiment, the invention provides an isolated nucleotide sequence having 75%-98% sequence identity to the nucleotide sequence set forth as SEQ ID NO: 86-114. In another embodiment, the invention provides an isolated nucleotide sequence having 80%-98%, 85%-98%, 87%-98%, 90%-98%, and 95%-98% sequence identity to the nucleotide sequence set forth as SEQ ID NO: 86-114. In another embodiment, the invention provides an isolated nucleotide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99% sequence identity to the nucleotide sequence set forth as SEQ ID NO: 86-114.

According to some embodiments, the invention provides an isolated nucleotide sequence selected from the group consisting of sequences set forth as SEQ ID NO: 86-114.

In one embodiment, the method of the invention utilizes the most comprehensive chimeric transcript repository, ChiTaRS 5.0 (http://chitars.md.biu.ac.il/), with 111,582 annotated entries from eight species. The repository includes unique information correlating chimeric breakpoints with 3D chromatin contact maps, generated from public datasets of chromosome conformation capture techniques (Hi-C). The repository comprises curated information on druggable fusion targets matched with chimeric breakpoints, which are applicable to precision medicine in cancers; as well as chimeric RNAs in various cell-lines and, novel chimeras in Alzheimer's disease, schizophrenia, dyslexia and other diseases. ChiTaRS stands out as a unique server that integrates EST and mRNA sequences, literature resources, with RNA-sequencing data, expression level and tissue specificity of chimeric transcripts in various tissues and organisms.

According to some embodiments, the method of cfDNA analysis is based on deep Illumina sequencing procedure as well as BGI sequencing and/or other deep sequencing procedures of cfDNA and/or circulating cell free RNA (cfRNA) extracted from patients' blood plasma (using dedicated Quiagen and/or other kits), followed by the efficient Bioinformatics analysis using in-house developed tool, method and apparatus.

As used herein, the term “therapeutic means” refers, without limitation, to the remedial agents or methods for the treatment of health-disease conditions or disorders. A non-limiting list of therapeutic means of the invention includes investigational drug, an approved drug, food supplement, phototherapy, radiation therapy, surgical intervention, hyperbaric oxygen, non-invasive image-guided procedures, multi-step treatment protocol, or any combination of the above. A non-limiting list of therapeutic means of the invention further includes probiotic-based therapeutic means, phage-based therapeutic means, small-molecule-based therapeutic means, prebiotic-based therapeutic means, clinical measures, mouth derived microbiome or any other clinically acceptable therapeutics. The therapeutic means of the invention can be, without limitation, newly discovered therapeutic means and/or known therapeutic means that are already in clinical use.

According to some embodiments, diagnostics associated with the condition can be assessed using one or more of: a behavioral survey instrument (e.g., a Patient Health Questionnaire-9 (PHQ-9) survey, a patient health questionnaire-2 (PHQ-2) survey, an instrument derived from an edition of the Diagnostic and Statistical Manual (DSM) of mental disorders, an instrument derived from the Social Communication Questionnaire (SCQ), a Clinical Global Impression (CGI) scale, a Brief Psychiatric Rating Scale, etc.); a motor skills based assessment, a blood cell analysis of a biological sample, imaging based method, stress testing, motion testing, biopsy, and any other standard method.

### EXAMPLES

In the examples below, if an abbreviation is not defined above, it has its generally accepted meaning.

### Example 1: Fusion Gene and Chimeric Transcripts Predicted in Neurodegenerative Disease Including Alzheimer's Disease and Dementia

The chimeric RNAs are predicted using 10 Alzheimer's disease brain samples and 10 Alzheimer's disease cell free DNA samples vs. 10 normal brain controls and 10 normal blood samples. Using our method, we identified unique fusions found only in Alzheimer's disease samples.

Reference is now made to Table 1, listing fusion gene and chimeric transcripts predicted in neurodegenerative disease including Alzheimer's disease and dementia.

TABLE 1

| SEQ. | | |
|---|---|---|
| ID. | | |
| NO. | GROUP | DESCRIPTION |
| 1 | Predicted chimeric transcript | COL3A1/RPL7AP30 |
| 2 | Predicted chimeric transcript | COL3A1/COL6A3 |
| 3 | Predicted chimeric transcript | CLU/ZBTB18 |
| 4 | Predicted chimeric transcript | CLU/ZBTB18_Junction2 |
| 5 | Predicted chimeric transcript | CLU/RNA45SN2 |
| 6 | Predicted chimeric transcript | COL3A1/AMOT |
| 7 | Predicted chimeric transcript | CLU/LOC101928307 |
| 8 | Predicted chimeric transcript | COL3A1/COL3A1 |
| 9 | Predicted chimeric transcript | CLU/CLU |
| 10 | Predicted chimeric transcript | COL3A1/MTCH1 |
| 11 | Predicted chimeric transcript | COL3A1/ND6 |
| 12 | Predicted chimeric transcript | COL3A1/DYNC1H1 |
| 13 | Predicted chimeric transcript | COL3A1/NUP50 |
| 14 | Predicted chimeric transcript | CLU/ATP6 |
| 15 | Predicted chimeric transcript | COL3A1/FST |
| 16 | Predicted chimeric transcript | COL3A1/HSH2D |
| 17 | Predicted chimeric transcript | CLU/MAP2K2 |
| 18 | Predicted chimeric transcript | CLU/RPS4X |
| 19 | Predicted chimeric transcript | CLU/FER1L4 |
| 20 | Predicted chimeric transcript | CLU/CDK9 |
| 21 | Predicted chimeric transcript | CLU/CLU_Junction2 |
| 22 | Predicted chimeric transcript | COL3A1/YWHAB |
| 23 | Predicted chimeric transcript | CLU/ZFAND2B |
| 24 | Predicted chimeric transcript | COL3A1/THBS2 |
| 25 | Predicted chimeric transcript | COL3A1/CHST12 |
| 26 | Predicted chimeric transcript | COL3A1/CCNI |
| 27 | Predicted chimeric transcript | COL3Al/COL3A1_Junction2 |
| 28 | Predicted chimeric transcript | COL3A1/CLU |
| 29 | Predicted chimeric transcript | COL3A1/COL3A1_junction3 |
| 30 | Predicted chimeric transcript | CLU/RNA45SN2_Junction2 |
| 31 | Predicted chimeric transcript | COL3A1/AHNAK |
| 32 | Predicted chimeric transcript | CLU/CHCHD2 |

### Example 2: Nucleosome-Derived Genomic Regions to Identify the Origin of Tumor

Glioblastoma cell line LN229, astrocytoma grade-IV cell line CCFSTTG1 and ovarian cancer cell line OVCAR3 were grown in-vitro. Nucleosomal bound DNA from the cell lines was obtained by the means of micro-coccal nuclease (MNase) digestion and cfDNA isolated from the culture media. Nucleosomal DNA fragments and media cfDNA were sequenced using the next generation sequencing platform and are screened for similar fragmentation pattern between the nucleosomal DNA and cfDNA obtained from the cells media of each cell line. The specificity of this fragmentation pattern is assessed by comparing the nucleosomal DNA and cfDNA of different cell lines.

Reference is now made to Table 2, listing unique hotspots on different human chromosomes that make profiling of the tissue origin using nucleosome positioning at these regions.

TABLE 2

| SEQ. | | |
|---|---|---|
| ID. | | CHR: |
| NO. | GROUP | GENOMIC LOCATION |
| 33 | Hotspot regions tissue of origin | 1: 125183000-125183050 |
| 34 | Hotspot regions tissue of origin | 1: 143213100-143213350 |
| 35 | Hotspot regions tissue of origin | 1: 143214250-143214900 |
| 36 | Hotspot regions tissue of origin | 1: 143232850-143233200 |
| 37 | Hotspot regions tissue of origin | 1: 143264550-143264700 |
| 38 | Hotspot regions tissue of origin | 2: 89831000-89831050 |
| 39 | Hotspot regions tissue of origin | 2: 89836450-89836600 |
| 40 | Hotspot regions tissue of origin | 2: 89841050-89841150 |
| 41 | Hotspot regions tissue of origin | 3: 75669100-75669400 |
| 42 | Hotspot regions tissue of origin | 5: 49658340-49658790 |
| 43 | Hotspot regions tissue of origin | 5: 49659390-49659640 |
| 44 | Hotspot regions tissue of origin | 5: 49661570-49661870 |
| 45 | Hotspot regions tissue of origin | 6: 157310800-157310950 |
| 46 | Hotspot regions tissue of origin | 6: 157314200-157314250 |
| 47 | Hotspot regions tissue of origin | 7: 59995750-59995850 |
| 48 | Hotspot regions tissue of origin | 8: 43237700-43237850 |
| 49 | Hotspot regions tissue of origin | 9: 42900300-42900450 |
| 50 | Hotspot regions tissue of origin | 10: 41859850-41860000 |
| 51 | Hotspot regions tissue of origin | 10: 133688150-133688850 |
| 52 | Hotspot regions tissue of origin | 10: 133689200-133689250 |
| 53 | Hotspot regions tissue of origin | 10: 133689350-133689500 |
| 54 | Hotspot regions tissue of origin | 10: 133689750-133689900 |
| 55 | Hotspot regions tissue of origin | 15: 17076150-17076200 |
| 56 | Hotspot regions tissue of origin | 16: 34588170-34588220 |
| 57 | Hotspot regions tissue of origin | 16: 46387830-46387980 |
| 58 | Hotspot regions tissue of origin | 16: 46389830-46390180 |
| 59 | Hotspot regions tissue of origin | 16: 46399420-46399520 |
| 60 | Hotspot regions tissue of origin | 17: 26884050-26884250 |
| 61 | Hotspot regions tissue of origin | 18: 108400-108500 |
| 62 | Hotspot regions tissue of origin | 19: 26161100-26161200 |
| 63 | Hotspot regions tissue of origin | 21: 7926350-7926450 |
| 64 | Hotspot regions tissue of origin | 22: 18896200-18896550 |

### Example 3: A List of Mitochondrial Fusions with Human Genomic DNA Found in Patients' Samples to Identify Tissue/s Hypoxia in Patients for the Personalized Hyper-Baric Oxygen Treatments

cfDNA is isolated and sequenced as previously described from biological samples of subjects afflicted with conditions associated with hypoxia. Reference is now made to Table 3, listing regions of the druggable mitochondrial fusions with human genome: that were observed in hypoxia conditions as a result of mitophagy, that is the selective degradation of mitochondria by autophagy processes. It often occurs to defective mitochondria following damage or stress or hypoxia of human cells.

TABLE 3

| SEQ. | | |
|---|---|---|
| ID. | | |
| NO. | GROUP | DESCRIPTION |
| 65 | Mitochondrial predicted kinase druggable | MT-ND5-MOB1A |
| | chimeras | |
| 66 | Mitochondrial predicted kinase druggable | AKAP12-ND1 |
| | chimeras | |
| 67 | Mitochondrial predicted kinase druggable | ND1-RACK1 |
| | chimeras | |
| 68 | Mitochondrial predicted kinase druggable | ATP8-MAPK3 |
| | chimeras | |
| 69 | Mitochondrial predicted kinase druggable | MT-RNR1-STRAP |
| | chimeras | |
| 70 | Mitochondrial predicted kinase druggable | TESK-1MT-ND2 |
| | chimeras | |
| 71 | Mitochondrial predicted kinase druggable | MT-RNR1-RACK1 |
| | chimeras | |
| 72 | Mitochondrial predicted kinase druggable | MT-RNR2-CAMK4 |
| | chimeras | |
| 73 | Mitochondrial predicted kinase druggable | CYTB-CDK19 |
| | chimeras | |
| 74 | Mitochondrial predicted chimeras | ATP8-ATP6 |
| 75 | Mitochondrial predicted chimeras | RNR2-ATP8 |
| 76 | Mitochondrial predicted chimeras | ND1-ND1 |
| 77 | Mitochondrial predicted chimeras | ND4-CLSPN |

### Example 4: A List of the Predicted Druggable Genomic DNA Fusions for Targeted Chemotherapy and Immune-Therapy Treatments for a Cancer Patient

CfDNA was isolated and sequenced as previously described from biological samples of cancer patients and normal controls. Reference is now made to Table 4 listing regions of the predicted druggable kinase genomic fusions.

TABLE 4

| SEQ. ID. | | |
|---|---|---|
| NO. | GROUP | DESCRIPTION |
| 78 | Predicted druggable kinases fusions | EWSR1/ERG |
| 79 | Predicted druggable kinases fusions | BCR/ABL1 |
| 80 | Predicted druggable kinases fusions | LOC105369736/LRRK2 |
| 81 | Predicted druggable kinases fusions | JAKMIP1/FLI1 |
| 82 | Predicted druggable genomic | BTK/CYB561D1 |
| | fusions | |
| 83 | Predicted druggable kinases fusions | SNAPC5/MAP2K1 |
| 84 | Predicted druggable kinases fusions | LOC105369782/ERBB3 |
| 85 | Predicted druggable kinases fusions | BCR/PDGFRA |

### Example 5: A List of the Druggable Kinase Fusions for Targeted Chemotherapy and Immune-Therapy Treatments for a Cancer Patient

25 cfDNA samples of cancer patients and 15 cfDNAs of healthy controls were analyzed. NGS analyses of cfDNA samples were produced to identify gene-gene fusions that found in cancer patients and absent in normal controls. Next, fusions that incorporate kinases and may be targeted by chemotherapy drugs using the prediction method (ChiPPI) were identified. All the druggable kinase fusions are summarized in Table 5.

TABLE 5

| SEQ. ID. | | |
|---|---|---|
| NO. | GROUP | DESCRIPTION |
| 86 | Druggable kinase fusions | RPL7A/NTRK1 |
| 87 | Druggable kinase fusions | TPR/MET |
| 88 | Druggable kinase fusions | PVT1/AKT3 |
| 89 | Druggable kinase fusions | SRGAP3/RAF1 |
| 90 | Druggable kinase fusions | ZDHHC1/ABL1 |
| 91 | Druggable kinase fusions | NTRK2/NTRK2 |
| 92 | Druggable kinase fusions | GOLPH3L/AKT3 |
| 93 | Druggable kinase fusions | RNA45SN2/KIT |
| 94 | Druggable kinase fusions | HSPB6/AKT1 |
| 95 | Druggable kinase fusions | MET/FAM3C2 |
| 96 | Druggable kinase fusions | CDKN2A/CDKN2A |
| 97 | Druggable kinase fusions | CDK4/ARV1 |
| 98 | Druggable kinase fusions | CDK4/JHY |
| 99 | Druggable kinase fusions | CDK12/CDK12 |
| 100 | Druggable kinase fusions | MAP2K1/SLC48A1 |
| 101 | Druggable kinase fusions | IGH/CDK12 |
| 102 | Druggable kinase fusions | AKT1/PPP3R1 |
| 103 | Druggable kinase fusions | CDK4/RPL4P4 |
| 104 | Druggable kinase fusions | SPTAN1/NTRK3 |
| 105 | Druggable kinase fusions | CDK4/IL1R1 |
| 106 | Druggable kinase fusions | NTRK3/NTRK3 |
| 107 | Druggable kinase fusions | NTRK3/ACTR8 |
| 108 | Druggable kinase fusions | BRAF/SPAG5-AS1 |
| 109 | Druggable kinase fusions | MALRD1/ATM |
| 110 | Druggable kinase fusions | CDK4/DNASE1 |
| 111 | Druggable kinase fusions | CDK4/AC007556.1 |
| 112 | Druggable kinase fusions | CNOT6L/AKT3 |
| 113 | Druggable kinase fusions | MAP2K1/PDK2 |
| 114 | Druggable kinase fusions | RAF1/MSS51 |

### Example 6: Fragment Size Estimation of Glioma Patients and Healthy Control's in Plasma cfDNA

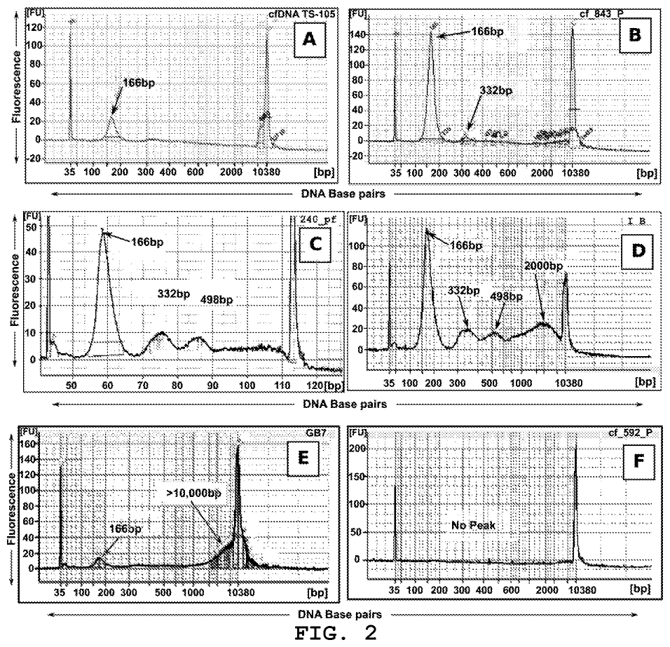

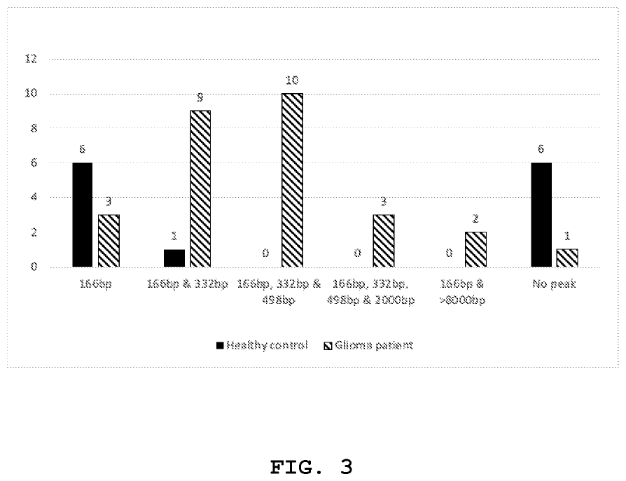

cfDNA fragment size distribution was examined in 41 samples obtained from glioma (n=27) patients and healthy (n=14) individuals by performing High Sensitivity Bioanalyzer DNA 1000 assay. As demonstrated on FIG. 2, electophoregrams of all 41 cfDNA samples showed mainly 6 types of DNA fragment enrichments. Amongst 9 out of 41 samples (2 GBMs, 1 LGG & 6 healthy), a major peak at ˜166 bp; in 10 out of 41 samples (9 GBMs, 1 healthy), a major peak at ˜166 bp and a small peak at ˜332 bp; and in 10 out of 41 samples (10 GBMs, 0 healthy), a major peak at ˜166 bp and smaller peaks at ˜332 bp and ˜498 bp were observed. While, 3 out of 41 samples (3 GBMs, 0 healthy) had a major peak at ˜166 bp and smaller peaks at ˜332 bp, ˜498 bp, and ˜2000 bp, and 2 out of 41 samples (2 GBMs, 0 healthy) had a major peak at ˜166 bp and enrichment at 10380 bp near upper reference ladder was observed. In the remaining 7 samples (1 LGG, 6 healthy) no fragment FIG. 3 is a graphical representation of DNA fragment sizes in healthy volunteers and gliomas. Black solid bars represent DNA fragment sizes in healthy volunteers, and striped bars represent DNA fragment sizes in glioma patients clearly showing an enrichment. The results are summarized in Table 6.

TABLE 6

| | Observed in | | | |
|---|---|---|---|---|
| DNA fragment size | samples out of | Healthy | Glioma | |
| Distribution | total 41 samples | Individuals | LGG | GBM |
| 166 bp | 9 | 6 | 1 | 2 |
| 166 bp, 332 bp | 10 | 1 | — | 9 |
| 166 bp, 332 bp, 498 bp | 10 | 0 | — | 10 |
| 166 bp, 332 bp, 498 bp, | 3 | 0 | — | 3 |
| 2000 bp | | | | |
| 166 bp, >8000 bp | 2 | 0 | — | 2 |
| No peak | 7 | 6 | 1 | — |

### DISCUSSION

These results indicate that GBM patient's plasma cfDNA show multiple fragment sizes such as 166 bp, 332 bp, 498 bp and 2000 bp; and healthy persons detectable plasma cfDNA shows mostly 166 bp fragments and rarely 332 bp fragments. Therefore, estimation of plasma cfDNA size enrichment may help in glioma liquid biopsy for distinguishing GBM patient's plasma cfDNA from healthy individuals.

### Example 7: High Fragmentation of Plasma cfDNA in GBM Patients than Healthy Controls

In this study, a high level of variable sizes cfDNA fragmentation (˜166 bp, ˜332 bp, and ˜498 bp) was observed in the GBM patients compared to healthy individuals, who had less cfDNA fragmentation.

Apoptotic fragmentation of cfDNA generates ˜166 bp, ˜332 hp, and ˜498 bp of reference DNA sizes. Amongst, ˜166 hp DNA is produced majorly, which is mononucleotide digestion equal to ˜147 bp of DNA wrapped around a nucleosome plus the stretch of DNA on Histone H1 linking two nucleosome cores. The longer fractions produced di-, tri-, or poly-nucleosomes nuclease action.

In three GBM patients, we observed the presence of 2000 bp DNA fragments along with the smaller fragments. The mechanism through which these fragments originate in the blood is still unknown. Less and consistent fragmentation of DNA in healthy controls and high and variable size fragmentation in the GBM patients underlines the use of studying fragmentation pattern as a marker in cancer screening and clinical outcome monitoring in the GBM patients was observed. Lastly, the observation of 8000 bp DNA fragments in two GBM patients represents DNA contamination. The plasma DNA fragments with size 8000 bp or more are typically referred to as the PBMCs genomic DNA contamination occurred during the plasma sample processing. PBMCs lysis occurred due to a lack of/insufficient preservation process, releases the large DNA fragments around 8000 bp in the sample. This genomic DNA contamination can be avoided by taking simple measures such as immediate processing of plasma after blood collection, in case of storage of blood sample, it can be stored for a maximum of 2 hours by keeping it on ice, and the separated plasma after centrifugation, if not used immediately, should be stored at −80° C. in the refrigerator.

Unless otherwise defined, all technical and/or scientific terms used herein have the same meaning as commonly understood by one of ordinary skill in the art, to which the invention pertains. Although methods and materials similar or equivalent to those described herein can be used in the practice or testing of embodiments of the invention, exemplary methods and/or materials are described below. In case of conflict, the patent specification, including definitions, will prevail. In addition, the materials, methods, and examples are illustrative only and are not intended to be necessarily limiting.

As used herein the terms “comprises”, “comprising”, “includes”, “including”, “having” and their conjugates mean “including but not limited to”.

The term “consisting of” means “including and limited to”.

As used herein, the singular form “a”, “an” and “the” include plural references unless the context clearly dictates otherwise. For example, the term “a compound” or “at least one compound” may include a plurality of compounds, including mixtures thereof.

It will be understood that when an element is referred to as being “on,” “attached” to, “connected” to, “coupled” with, “contacting,” etc., another element, it can be directly on, attached to, connected to, coupled with and/or contacting the other element or intervening elements can also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature can have portions that overlap or underlie the adjacent feature.

It will be understood that, although the terms first, second, etc., may be used herein to describe various elements, components, regions, layers and/or sections, these elements, components, regions, layers and/or sections should not be limited by these terms. Rather, these terms are only used to distinguish one element, component, region, layer and/or section, from another element, component, region, layer and/or section.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

As used herein, the term “non-linear reads” refers to nucleotide sequences which do not map linearly to the target genome. A non-limiting list of non-linear reads of the invention includes genomic integrations; exon-exon combinations; exon-intron combinations and/or any other sequence parts merged together

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

By “patient” or “subject” is meant to include any mammal. A “mammal,” as used herein, refers to any animal classified as a mammal, including but not limited to, humans, experimental animals including monkeys, rats, mice, and guinea pigs, domestic and farm animals, and zoo, sports, or pet animals, such as dogs, horses, cats, cows, and the like.

“Treating” or “treatment” of a disease as used herein includes: preventing the disease, i.e. causing the clinical symptoms of the disease not to develop in a mammal that may be exposed to or predisposed to the disease but does not yet experience or display symptoms of the disease; inhibiting the disease, i.e., arresting or reducing the development of the disease or its clinical symptoms, or relieving the disease, i.e., causing regression of the disease or its clinical symptoms, and/or monitoring the disease and early diagnostics of the disease.

Druggability, is a term used in drug discovery to describe a biological target such as a protein that is known to bind or is predicted to bind with high affinity to a drug. Furthermore, the binding of the drug to a druggable target alters the function of the target with a therapeutic benefit to the patient. The term “drug” herein includes small molecules (low molecular weight organic substances) but also has been extended to include biologic medical products such as therapeutic monoclonal antibodies. In at least one embodiment, the gene fusion or gene variant can be used to identify a druggable target.

Certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

1.-18. (canceled)

19. A method for treating a condition in a subject, wherein said condition is characterized by omics-discoverable features, the method comprising:
a. obtaining a biological sample from at least one subject, wherein said biological sample comprises cell-free nucleic acids;
b. sequencing said cell-free nucleic acids;
c. mapping the sequencing results to the reference human genome;
d. identifying unmapped non-linear reads;
e. mapping the unmapped non-linear reads to the pre-computed sequence data set indicative of said condition;
f. identifying at least one sequence associated with said condition;
g. applying a pre-computed treatment model to identify therapeutic means suitable for treating the condition; h. identifying the therapeutic means for treating the condition based on the pre-computed treatment model; and i. providing the human subject with the therapeutic means to thereby effectively treat the condition in the human subject.

20. The method of claim 19, further comprising the step of isolating circulating cell-free nucleic acids from the biological sample.

21. The method of claim 19, wherein the biological sample is selected from the group consisting of blood, serum, plasma, urine, saliva, amniotic fluid, feces, synovial fluid, peritoneal fluid, tissue biopsy, pleural fluid, lymphatic fluid, mucus, and cerebrospinal fluid (CSF).

22. The method of claim 21, wherein the biological sample is a liquid biological sample.

23. The method of claim 19, wherein the circulating cell-free nucleic acids is selected from the group consisting of circulating cell-free RNA, circulating cell-free DNA, circulating cell free nucleic acid complexes, and circulating cell-free microRNA.

24. The method of claim 19, wherein the condition characterized by omics-discoverable features is a neurodegenerative or a malignant disorder.

25. The method of claim 24, wherein the neurodegenerative disorder is selected from the group consisting of Alzheimer's disease (AD), dementia, Parkinson's disease (PD), PD-related disorders, Prion disease, Motor neuron diseases (MND), Huntington's disease (HD), Spinocerebellar ataxia (SCA), and Spinal muscular atrophy (SMA).

26. The method of claim 24, wherein the malignant disorder is selected from the group consisting of sarcoma, carcinoma, melanoma, glioma, glioblastoma, lymphoma, astrocytoma, Grade I—pilocytic astrocytoma, Grade II—Low-grade astrocytoma, Grade III—anaplastic astrocytoma, chordoma, CNS lymphoma, craniopharyngioma, brain stem glioma, ependymoma, medulloblastoma, and meningioma.

27. The method of claim 19, wherein the at least one sequence associated with said condition is selected from the group consisting of sequences having at least 75% sequence identity to the nucleotide sequence set forth as SEQ ID NO:1-32.

28. The method of claim 19, wherein the at least one sequence associated with said condition is selected from the group consisting of sequences having at least 75% sequence identity to the nucleotide sequence set forth as SEQ ID NO: 33-64.

29. The method of claim 19, wherein the at least one sequence associated with said condition is selected from the group consisting of sequences having at least 75% sequence identity to the nucleotide sequence set forth as SEQ ID NO:65-77.

30. The method of claim 19, wherein the at least one sequence associated with said condition is selected from the group consisting of sequences having at least 75% sequence identity to the nucleotide sequence set forth as SEQ ID NO:78-85.

31. The method of claim 19, wherein the at least one sequence associated with said condition is selected from the group consisting of sequences having at least 75% sequence identity to the nucleotide sequence set forth as SEQ ID NO:86-114.

32. The method of claim 19, wherein the at least one sequence associated with said condition is selected from the group consisting of the nucleotide sequences set forth as SEQ ID NO:1-114.

33. The method of claim 19, wherein omics-discoverable features are selected from the group consisting of genomics-discoverable features, proteomics-discoverable features, metagenomics-discoverable features, methylomics-discoverable features, epigenomics-discoverable features, microbiome-discovered features, hypoxia-discoverable features, and metabolomics-discoverable features.

34. The method of claim 19, wherein omics-discoverable features are selected from chimeras, chimeric RNAS, gene-gene fusions, sense-antisense (SAS) chimeras, Exon-intron fusions, exon-exon fusions, intron-exon fusions, genomic integrations, aberrations, pathogen integrations and inversions.

35. The method of claim 19, wherein the therapeutic means are selected from the group consisting of an investigational drug, an approved drug, a food supplement, phototherapy, radiation therapy, surgical intervention, hyperbaric oxygen, non-invasive image-guided procedure, multi-step treatment protocol, or any combination thereof.

36.-49. (canceled)

50. An isolated nucleotide sequence having at least 75% sequence identity to a nucleotide sequence selected from the group consisting of SEQ ID NO:1-114.

51.-54. (canceled)

55. An isolated nucleotide sequence selected from the group consisting of nucleotide sequences set forth as SEQ ID NO: 1-114.

56-58. (canceled)


SEQUENCE LISTING (114 sequences)

SEQ ID NO: 1 - DNA, 200 bp, Artificial
  Synthetic
  misc_feature: predicted chimeric transcript
  misc_feature: COL3A1/RPL7AP30
cacaaagagtctcatgtctgatatttagacatgatgagctttgtgcaaaaggggagctggctacttctcgctctgcttcatcccactattattttggcacaacaggaagacctgcaccactgtcgccttcacacaggtgaactcggaagacaaaggcgcttaggctaagctggtggaagctatcaggatcaattacaatt

SEQ ID NO: 2 - DNA, 280 bp, Artificial
  Synthetic
  misc_feature: COL3A1/COL6A3
  misc_feature: predicted chimeric transcript
gttatcataaagagtgttgaagtttatttattatagcaccattgagacattttgaaattggaattggtaaaaaaataaaacaaaaagcatttgaattgtatttggtggaacagcaaaaaaagagaagtatcatttttctttgtcaaattatactgtttccaaacattttggaaataaataactggaattttgtcggtcactgggttggatgtaggacttgaagtaacgttattcggaacatttacttgttttgtgaccaaacttcacaaggttctttgtg

SEQ ID NO: 3 - DNA, 493 bp, Artificial
  Synthetic
  misc_feature: CLU/ZBTB18
  misc_feature: Predicted chimeric transcript
tatcagctcacaagacagttttattgaattagttgcatgcaggagcaattctgttcttcccatgagcagcagagtcgagtgttagagtgcaggatgttcaatttttcatcttctccttcatcttcatcactgggcaaatcgcctgctatgtggctgctgccatcggagagactctcgactttgtccgaacaacttgaagcatcttcttcctttttggtgctgtctgcctccgtggtggctttctctttcagcttctttttgcagactttgacaatgtcatacatgtggagataactggcagctgctagcacgtcttcaatgggcaagtctttgaactggagtttcccttcatacatgaattcaagcaggagagcgaaagcgggggttgtaacaatgtcgctgttcagatgaacaatgtctcttttgtccagctggtccttgtaaaagaggtggaaatacatgctgcatgaagccagtacagctcggtgcgctc

SEQ ID NO: 4 - DNA, 511 bp, Artificial
  Synthetic
  misc_feature: CLU/ZBTB18_Junction2
  misc_feature: Predicted chimeric transcript
ttatcagctcacaagacagttttattgaattagttgcatgcaggagcaattctgttcttcccatgagcagcagagtcgagtgttagagtgcaggatgttcaatttttcatcttctccttcatcttcatcactgggcaaatcgcctgctatgtggctgctgccatcggagagactctcgactttgtccgaacaacttgaagcatcttcttcctttttggtgctgtctgcctccgtggtggctttctctttcagcttctttttgcagactttgacaatgtcatacatgtggagataactggcagctgctagcacgtcttcaatgggcaagtctttgaactggagtttcccttcatacatgaattcaagcaggagagcgaaagcgggggctgtaacaatgtcgctgttcagatgaacaatgtctcttttgtccagctggtccttgtaaaagaggtggaaatacatgctgcatgaagccagtacagctcggtgcgctcggaactgggcatctccc

SEQ ID NO: 5 - DNA, 292 bp, Artificial
  Synthetic
  misc_feature: CLU/RNA45SN2
  misc_feature: Predicted chimeric transcript
ttcagctcacaagacagttttattgaattagttgcatgcaggagcaattctgttcttcccatgagcagcagagtcgagtgttagagtgcaggatatgggtacggcccggcgcgagatttacaccctctcccccggattttcaagggccagcgagagctcaccggacgccgccggaaccgcgacgctttccaaggcacgggcccctctctcggggcgaacccattccagggcgccctgcccttcacaaagaaaagagaactctccccggggctcccgccggcttctccgggat

SEQ ID NO: 6 - DNA, 487 bp, Artificial
  Synthetic
  misc_feature: COL3A1/AMOT
  misc_feature: Predicted chimeric transcript
gggctgagttttatgacgggcccggtgctgaagggcagggaacaacttgatggtgctactttgaactgcttttcttttctcctttttgcacaaagagtctcatgtctgatatttagacatgatgagctttgtgcaaagggggagctggctacttctcgctctgcttcatcccccttggtcccaaaccagggagaccattcagctcacctgcctaggccgcagcagcatttccttcctaatcaggctcaccagggggatcattaccgtctctcccaacctggcctgagtcagcagcagcagcaacagcagcagcagcaccatcatcaccatcaccaccaacaacagcagcagcagcagccacagcagctgccgggagaagcctattcagctatgcctcgggctcagccatcctctgcttcttatcagccagtgccagcagacccttttgccattgtttccagagcccagcagatggttgagatcctct

SEQ ID NO: 7 - DNA, 490 bp, Artificial
  Synthetic
  misc_feature: CLU/LOC101928307
  misc_feature: Predicted chimeric transcript
tttttttttttagctcacaagacagttttattgaattagttgcatgcaggagcaattctgttcttcccatgagcagcagagtcgagtgttagagtgcaggatccagagcggggagaggaataaaaatggaaaggactgtctgtacctcaaaagatgatagcaagggagaaaggcaaagctagtaaaactatggaggttttcaaaattgtgatagaatagagaggcatttccaacctgtgagtgggaaaattgtgattctttctagtctaggatttaattgcatttttctctcactgcttccatttcttgtgagacaccttctgttatttagactatgtcaggaagagccttagacattattcaatcaagctcttttctgtgagataactgaagtccagagaattcatagcttcttgtccttaatgaggtatatacttagtgtcagtttcgtgagtaaaaatgaagaatcttgatttctagtataattgaa

SEQ ID NO: 8 - DNA, 169 bp, Artificial
  Synthetic
  misc_feature: COL3A1/COL3A1
  misc_feature: Predicted chimeric transcript
ggagcaaaacagtctttgaatatcgaacacgcaaggctgtgagactacctattgtagatattgcaccctatgacattggtggtcctgatcaagaatttgttgggatttcagatagagtttggtttataaaaagcaaacagggccaacgtccacaccaaattcttgatca

SEQ ID NO: 9 - DNA, 291 bp, Artificial
  Synthetic
  misc_feature: CLU/CLU
  misc_feature: Predicted chimeric transcript
taaattcgcggccgcgtcgactttttttttttttttttttttttcagctcacaagacagttttattgaattagttgcatgcaggagcaattctgttcttcccatgagcagcagagtcgagtgttagagtgcaggatccaaagcggggagaggctgggcggaattgggggcctggaggctggggcctggttacttggtgacgtgcagagctctctctggatgttgcttttgcacctacgggggcatctgagtccagctccccccaagatgagctgcagccccccagagagag

SEQ ID NO: 10 - DNA, 608 bp, Artificial
  Synthetic
  misc_feature: COL3A1/MTCH1
  misc_feature: Predicted chimeric transcript
cacgagcaggccctgctggtgttcctgggagaaatggtgaccctggtattccaggacaaccagggtcccctggttctcctggcccccctggaatctgtgaatcatgccctactggtcctcagaactattctccccagtatgattcatatgattcactgctggaagtacctgagtgtgcagggccagctcttccgaggcttcagcctgcttttccgccgggtgtcatcaggatcatgctttgccctggagtaacctgaatcatttaaaaaacacggtctcaacctggccaccgagggtgaggcctgaccaccttgggacacctgcaagactactccaacccaacatcaaccagatgtgctccaacccagccgggcttcagttccatatttgccatgtgtcttgtccagatgtggggtttgagcgggggtggggctgcaccccatggattgggtcacccggcagtacctaggaaatgtaaaggctttggtggggtatttcctaaaattttctgtcctttaataattttcttatttatataatattactcaggtgtgagggctagattctctgattgtccatttttgtgtggggttaaaacatatacttgg

SEQ ID NO: 11 - DNA, 397 bp, Artificial
  Synthetic
  misc_feature: COL3A1/ND6
  misc_feature: Predicted chimeric transcript
attctgtcactggcactggttgacaagattagaacaagaggaacacatatggagttaaattttttgtgggatttcagatagagttggtttataaaaagcaaacagggccaacgtccacaccaaattcttgatcaggaccaccaatgtcatagggtgcaatatctacaataggtagtctcacagccttgcgtgttcgatattcaaagactgtttcgctccattccccagtgtgtttcgtgcaaccatcctccagaactgtgtaggtgaatttgctatttccttcagccttgacccctctccttcataaattattcagcttcctacactattaaagtttaccacaaccaccaccccatcatactctttcacccacagcaccaatcctacctccagcgct

SEQ ID NO: 12 - DNA, 700 bp, Artificial
  Synthetic
  misc_feature: COL3A1/DYNC1H1
  misc_feature: Predicted chimeric transcript
cgggtactggttgaccctaaccaaagatgcaaattggatgctatcaaggatattctgtaatatggaaactggggaaacatgcataagtgccaatcctttgaatgttccacggaaacactggtggacagattctagtgctgagaagaaacacgtttggtttggagagtccatggatggtggttttcagtttagctacggcaatcctgaacttcctgaagatgtccttgatgtgcagctggcattccttcgacttctctccagccgagcttcccagaacatcacatatcactgcaaaaatagcattgcatacatggatcaggccagtggaaatgttcacagaacaacaccagccactttccaaattgaacaggagccaaaaccaccccattaggtgtgcgcctgtactcgcagtagtgatcaaaagtcttcagaagcagctctggagtagtagcactggagaagttgagacccaccacctccatgtcaggcaaggcccggagggcgctgaagagtgtcatggtcttgccagacccaggagggccacacaagaccaggggcttgtgttcggccagccaagtgtacaagagggcttcgtggcggactgtgtccagcgttggcacgacgacatcaggggctgccaccttgtgcgtctccacttcaatctgaggcaccttggcctgccacggaagacattctccgc

SEQ ID NO: 13 - DNA, 317 bp, Artificial
  Synthetic
  misc_feature: COL3A1/NUP50
  misc_feature: Predicted chimeric transcript
taagcgcgtatccgaatattagtatcttgacagcaatgacaagaaaaaagaggagcaccattacaagcagttgatagctctgttttgggctcattaagctctgtccccctgactggatttctttctcccctggaaactccagtatattttggcatagatactacccagagtaaaccagtctcttcaccatttcccactaaaccattggagggccaagcagaaggtgacagcggtgaatgcataggtggagatgaagaagagaatgatgggccacccaaagtaatagttaccgaagtaaaagaagaaggcgctgtagg

SEQ ID NO: 14 - DNA, 345 bp, Artificial
  Synthetic
  misc_feature: CLU/ATP6
  misc_feature: Predicted chimeric transcript
actccacagcccggcttccagcacccgccaacagaattcatacgagaaggcgacgaggaccggacgggtgccgggagatccgccacaactccacgggtcgcctgcggatgaaggaccagtgtgacgagtgccgggagatcttgtctgtggactgttccaccaacaacccctcccaggctaagctgcggcgggagctcgacgaatcaaactaacctcaaaacaaatgataaccatacacaacactaaaggacgaacctgatctcttatactagtatccttaatcatttttattgccacaactaacctcctcggactcctgcctcactcatttacaccaaccaccca

SEQ ID NO: 15 - DNA, 486 bp, Artificial
  Synthetic
  misc_feature: COL3A1/FST
  misc_feature: Predicted chimeric transcript
cgagaagatcacctccagaacgtgtaggtgaattgctatttccttcagccttgaattcaccttcattgaccccatcagcttcaggggccttctttacatttccactggcctgatccatgtatgcaatgctatttttgcagtgatatgtgatgttctgggaagctcggctggagagaagtcgaaggaatgccagctgcacatcaaggacatcttcaggaagctcaggattgccgtagctaaactgaaaaccaccatccatggactctccaaaccaaacgtgtttcttctcagcactagaatctgtccaccagtgtttccgtggaacattcaaaggattggcacttatgcatgtttccccagtttccatattacagaataccttgatagcatccaatttgcatccttggttgaggaaactcctacttcagttctttggttatgatgaagtcatttgtgagagaggtgatctaaggtgatcttctgacg

SEQ ID NO: 16 - DNA, 454 bp, Artificial
  Synthetic
  misc_feature: COL3A1/HSH2D
  misc_feature: Predicted chimeric transcript
acgggtgatttttacgagaaccatcaggactaatgaggctttctatttgtccattaacagacttgagtgaagtcataatctcatcggtgttgattttgaaatccattggttcatctccataatacggggcaaaaccgccagctttttcacctccaatcccagcaatggcagcggctccaacaccaccacagcaaggaccaggggcaccaggaggtccaggagggcctggttgccctgtgtggccacatctctcaaaagcccctcacagccccaggcaccaaaagacagaaaggtccccaccaggaaggccgagaggtcggtcagctgcattgaggtgaccccagtggacaggagttggcaccaaatggtagtgagagccctatcctcccaggagtccaagccagagcaccagggcttggcagagcctgagaacgaccagctcccggaggagggg

SEQ ID NO: 17 - DNA, 530 bp, Artificial
  Synthetic
  misc_feature: CLU/MAP2K2
  misc_feature: Predicted chimeric transcript
gcaaggactccatgaattggagcatgatgaagactctgctgctgtttgtggggctgctgctgacctgggagagtgggcaggtcctgggggaccagacggtctcagacaatgagctccaggaaatgtccaatcagggaagtaagtacgtcaataaggaaattcaaaatgctgtcaacggggtgaaacagataaagactctcatagaaaaaacaaacgaagagcgcaagacactgctcagaaacctgcgcagagcctgcactcactccttgtgtgccctctaggacggcggctccctggaccaggtgctgaaagaggccaagaggattcccgaggagatcctggggaaagtcagcatcgcggttctccggggcttggcgtacctccgagagaagcaccagatcatgcaccgagatgggaagccctccaacatcctcgtgaaactctagaggggagatcaagctgtgtgacttcggggtgagcggccagctcatcgactccatggccaactccttcgtgggcacgcggctcct

SEQ ID NO: 18 - DNA, 539 bp, Artificial
  Synthetic
  misc_feature: CLU/RPS4X
  misc_feature: Predicted chimeric transcript
gtctccataaatagggtcttcctggagacttctacagggaccgtcacagtgatgggatcagagtcaaagagcttcacgaccacctcagtgacaccggaaggaacgtccgagtcagaagtgtgggaagccaccgtggtgacccgcagatagtactggtcttcgccttgcgtgaggtttgccagccgggacacccagttaaactgctcgttcaactgctccagcaaggaggaggtgttgagcatcttccactggtaggactttagcagctcgttgtatttcctggtcaacctctcagcgacctggagggattcgtcgagctcccgccgcagcttagcctgggaggggttgttggtggaacagtccacagacaagatctcccggcacttgtcacactggtccttcatccgcaggcagcccgtggagttgtggcggatctcccggcacacagtccggtcatcgagagaggcaccctggatcttttgacgtggttcacgtgaaagatgccaatggcaacagctttgccactcgactttccaaca

SEQ ID NO: 19 - DNA, 207 bp, Artificial
  Synthetic
  misc_feature: CLU/FER1L4
  misc_feature: Predicted chimeric transcript
gctctggatcctgcactctaacactcgactctgctgctcatgggaagaacagaatggctcctgcatgcattgaagggttggcaccctgggtgagtgtccaaggtcagcgagagtcacttgtggaggggacggaagatgacctggctgatctggccagggatggtgtagaagaccaggaggaggacagacggtgagaagcactcggag

SEQ ID NO: 20 - DNA, 182 bp, Artificial
  Synthetic
  misc_feature: CLU/CDK9
  misc_feature: Predicted chimeric transcript
gttgaccaggaaatacaacgagctggtaaagtcctaccagtggaagatgctcaacacctcctccttgctggttttctggatggttcccagagggtttccatggggtaggaggatgggctcgcccaccagtgactttttctaagagctcccggcgtggtggaagaggggacaggtccctcact

SEQ ID NO: 21 - DNA, 226 bp, Artificial
  Synthetic
  misc_feature: CLU/CLU_Junction2
  misc_feature: Predicted chimeric transcript
tctagaggatcccaagctctggaaaaggggtgaaacagactaagactctcatagaaaaaacaaacgaagagcgcaagacactgctcagcaacctagaagaagccaagaagaagaagtgaggcctccggtggggcaggctgaagggcaggtagtggtaggtatcctggggctcccgggtgaagaacctgtcctggaagagctcgtctatgatgctggacgcgcggct

SEQ ID NO: 22 - DNA, 755 bp, Artificial
  Synthetic
  misc_feature: COL3A1/YWHAB
  misc_feature: Predicted chimeric transcript
ttttttttttttttttatcataaagagtgttgaagtttatttattatagcaccattgagacattttgaaattggaattggtaaaaaaataaaacaaaaagcatttgaattgtatttggtggaacagcaaaaaaagagaagtatcatttttctttgtcaaattatactgtttccaaacattttggaaataaataactggaattttgtcggtcacttgcactggttgacaagattagaacaagaggaacacatatggagttaaattttttttgttgggatttcagatagagtttggtttataaaaagcaaacagggccaacgtccacaccaaattcttgatcaggaccaccaatgtcatagggtgcaatatctacaataggtagtctcacagccttgcgtgttcgatattcaaagactgttttgctccattccccagtgtgtttcgtgcaaccatcctccagaactgtgtaggtgaatttgctatttccttcagccttgaattcaccttcatttgaccccatcagcttcatgcccctgttctgtgactgccttcatggctgcagccatatcatcatatcgctcagcctgctcagcgagtttggctttctgtaccagctcacttttatccattgtcattcccgtgggagggctgcagggaacaaaacccttccgagcgaactccgagcgagatgactcttccttcctaagccactactcaagcggcggcagcggtgcctgcagtggctgcctcgtgcc

SEQ ID NO: 23 - DNA, 190 bp, Artificial
  Synthetic
  misc_feature: CLU/ZFAND2B
  misc_feature: Predicted chimeric transcript
agccgcgcgtccagcatcatagacgagctcttccaggacaggttcttcacccgggagccccaggatacctaccactacctgccctccagggtgctccgggcctcctctccctgtgtaccctagaggtgtgagggccacagtcctcctcaggtgaacctccctccccaagcccagggccctagcacaggct

SEQ ID NO: 24 - DNA, 249 bp, Artificial
  Synthetic
  misc_feature: COL3A1/THBS2
  misc_feature: Predicted chimeric transcript
atcataaagagtgttgaagtttatttattatagcaccattgagacattttgaaattggaattggtaaaaaaataaaacaaaaagcatttgaattgtatttggtggaacagcaaaaaaagagaagtatcatttttcaaatatttatttatgtgttcacatggtcaaaatttcaccactgaaaccctgcacttagctagaacctcatttttaaagattaacaacaggaaataaattgtaaaavvggttttc

SEQ ID NO: 25 - DNA, 777 bp, Artificial
  Synthetic
  misc_feature: COL3A1/CHST12
  misc_feature: Predicted chimeric transcript
aggttctggacctgctggtcctcctggtccccaaggtgtcaaaggtgaacgtggcagtcctggtggacctggtgctgctggcttccctggtgctcgtggtcttcctggtcctcctggtagtaatggtaacccaggacccccaggtcccagcggttctccaggcaaggatgggcccccaggtcctgcgggtaacactggtgctcctggcagccctggagtgtctggaccaaaaggtgatgctggccaaccaggagagaagggatcgcctggtgcccagggcccaccaggagctccaggcccacttgggattgctgggatcactggagcacggggtcttgcaggaccaccaggcatgccaggtcctaggggaagccctggccctcagggtgtcaagggtgaaagtgggaaaccaggagctaacggtctcagtggagaacgtggtccccctggaccccagggtcttcctggtctggctggtacagctgggccggcagcaggcggagcggaggagcgtgctgcggggcttctgcgccaactccagcctggccttccccaccaaggagcgcgcattcgacgacatccccaactcggagctgagccacctgatcgtggacgaccggcacggggccatctactgctacgtgcccaaggtggcctgcaccaactggaagcgcgtgatgatcgtgctgagcggaagcctgctgcaccgcggtgcgccctaccgcgacccgctgcgcatcccgcgcgagcacgtgcacaacgccagcgcgcacctga

SEQ ID NO: 26 - DNA, 558 bp, Artificial
  Synthetic
  misc_feature: COL3A1/CCNI
  misc_feature: Predicted chimeric transcript
ttttttttatcataaagagtgttgaagtttatttattatagcaccattgagacattttgaaattggaattggtaaaaaaataaaacaaaaagcatttgaattgtatttggtggaacagcaaaaaaagagaagtatcatttttctttgtcaaattatactgtttccaaacattttggaaataaataactggaattttgtcggtcacttgcactggttgacaagattagaacaagaggaacacatatggagttaaatttttttgttgggatttcagatagagtttggtttataaaaagcaaacagggccaacgtccacaccaaattcttgatcaggaccaccaatgtcatagggtgcaatatctacaataggtagtctcacagccttgcgtgttcgatattcaaagactgttttgctccattccccagccttatgtttcttttaatgacttctggatcctgagctccctttgcagtctgaaaaaggtattgcagtcagaactgtgtactgatgataaaagcctctggtagcaataaaaagttgtccttaaaaaaaaaaaa

SEQ ID NO: 27 - DNA, 670 bp, Artificial
  Synthetic
  misc_feature: COL3A1/COL3A1_Junction2
  misc_feature: Predicted chimeric transcript
tgaaaaccaccatccatggactctccaaaccaaacgtgtttcttctcagcacatcacatatcactgcaaaaatagcattgcatacatggatcaggccagtggaaatgtaaagaaggccctgaagctgatggggtcaaatgaaggtgaattcaaggctgaaggaaatagcaaattcacctacacagttctggaggatggttgcacgaaacacactggggaatggagcaaaacagtctttgaatatcgaacacgcaaggctgtgagactacctattgtagatattgcaccctatgacattggtggtcctgatcaagaatttggtgtggacgttggccctgtttgctttttataaaccaaactctatctgaaatcccaacaaaaaaaatttaactccatatgtgttcctcttgttctaatcttgtcaaccagtgcaagtgaccgacaaaattccagttatttatttccaaaatgtttggaaacagtataatttgacaaagaaaaatgatacttctctttttttgctgttccaccaaatacaattcaaatgctttttgttttatttttttaccaattccaatttcaaaatgtctcaatggtgctataataaataaacttcaacactctttatgataacaacactgtgttatattctttgaatcctaccccatct

SEQ ID NO: 28 - DNA, 725 bp, Artificial
  Synthetic
  misc_feature: COL3A1/CLU
  misc_feature: Predicted chimeric transcript
ggagattctagtgctgagaagaaacacgtttggtttggagagtccatggatggtggttttcagtttagctacggcaatcctgaacttcctgaagatgtccttgatgtgcagctggcattccttcgacttctctccagccgagcttcccagaacatcacatatcactgcaaaaatagcattgcatacatggatcaggccagtggaaatgtaaagaaggccctgaagctgatggggtcaaatgaaggtgaattcaaggctgaaggaaatagcaaattcacctacacagttctggaggatggttgcacgaaacacactggggaatggagcaaaacagtctttgaatatcgaacacgcaaggctgtgagactacctattgtagatattgcaccctatgacattggtagcctgccccaccggaggcctcacttcttctttcccaagtcccgcatcgtccgcagcttgatgcccttctctccgtacgagcccctgaacttccacgccatgttccagcccttccttgagatgatacacgaggctcagcaggccatggacatccacttccatagcccggccttccagcacccgccaacagaattcatacgagaaggcgacgatgaccggactgtgtgccgggagatccgccacaactccacgggctgcctgcggatgaaggaccagtgtgacaagtgccgggagatcttgtctgtggactgttccaccaacaa

SEQ ID NO: 29 - DNA, 615 bp, Artificial
  Synthetic
  misc_feature: COL3A1/COL3A1_junction3
  misc_feature: Predicted chimeric transcript
ggtactcctggtctgcaaggaatgccagcagcaccaggtggcccaggaggaccagcagcaccctttcctccttcgggaccagggggaccagttccacctggtgctgctggtactcctggtctgcaaggaatgcctggagaaagaggaggtcttggaagtcctggtccaaagggtgacaagggtgaaccaggcggtccaggtgctgatggtgtcccagggaaagatggcccaaggggtcctactggtcctattggtcctcctggcccagctggccagcctggagataagggtgaaggtggtgcccccggacttccaggtatagctggacctcgtggtagccctggtgagagaggtgaaactggccctccaggacctgctggtttccctggtgctcctggacagaatggtgaacctggtggtaaaggagaaagaggggctccgggtgagaaaggtgaaggaggccctcctggagttgcaggaccccctggaggttctggacctgctggtcctcctggtccccaaggtgtcaaaggtgaacgtggcagtcctggtggacctggtgctgctggcttccctggtgctcgtggtcttcctggtcctcctggtagtaatggt

SEQ ID NO: 30 - DNA, 381 bp, Artificial
  Synthetic
  misc_feature: CLU/RNA45SN2_Junction2
  misc_feature: Predicted chimeric transcript
actttagcagctcgttgtatttcctggtcaacctctcagcgacctggagggattcgtcgagctcagggaggacagaaacctcccgtggagcagaagggcaaaagctcgcttgatcttgattttcagtacgaatacagaccgtgaaagcggggcctcacgatccttctgaccttttgggttttaagcaggaggtgtcagaaaagttaccacagggataactggcttgtggcggccaagcgttcatagcgacgtcgctttttgatccttcgatgtcggctcttcctatcattgtgaagcagaattcaccaagcgttggattgttcacccactaatagggaacgtgagctgggattagaccgtcgtgagacaggttagttttac

SEQ ID NO: 31 - DNA, 538 bp, Artificial
  Synthetic
  misc_feature: COL3A1/AHNAK
  misc_feature: Predicted chimeric transcript
ttttcttttctcctttttgcacaaagagtctcatgtctgatatttagacatgatgagctttgtgcaaaaggggagctggctacttctcgctctgcttcatcccactattattttggcacaacaggaagctgttgaaggaggatgttcccatcttggtcagtcctatgccggatagagatgtctggaagccagaaccatgccaaatatgtgtctgtgactcaggatccgttctctgcgatgacataatatgtgacgatcaagaattagactgccccaacccagaaattccatttggagaatgttgtgcagtctgcccacagcctccaactgctcctactcgccctcctaatggtcaaggacctcaaggcgaaggctcccaaggtcaagatgccagatgtggacatctcagtgccaaaaatagagggtgacctgaaaggccccaaagtgcaggcaaacttgggtgcacctgacatcaacatcgaaggcctagatgctaaagtcaaaacaccgtccttcggcatttctgcccctcaagtct

SEQ ID NO: 32 - DNA, 499 bp, Artificial
  Synthetic
  misc_feature: CLU/CHCHD2
  misc_feature: Predicted chimeric transcript
ggcgtcgccaggaggagcgcgcgggcacagggtgccgctgaccgaggcgtgcaaagactccagaattggaggcatgatgaagactctgctgctgtttgtggggctgctgctgacctgggagagtgggcaggtcctgggggaccagacggtctcagacaatgagctccaggaaatgtccaatcagggaagtaagtacgtcaataaggaaattcaaaatgctgtcaacggggtgaaacagataaagactctcatagaaaaaacaaacgaagagcgcaagacactgctcagcaacctagaagaagccaagaagaagaaagaggatgccctaaatgagaccagggaatcagagacaaagctgaaggagacatcaagctctgtgagggtttcaatgaggtgctgaaacagtgccgacttgcaaacggattggcctaatgaagaagttcaacctggagagatggaaaatcagctctcataactaagttaatttagtataaaaata

SEQ ID NO: 33 - DNA, 50 bp, Homo sapiens
  misc_feature: 1125183000-125183050
  misc_feature: Hotspot regions tissue of origin
attccatttgatgttgattccattcgattccattttatgatgattccatg

SEQ ID NO: 34 - DNA, 250 bp, Homo sapiens
  misc_feature: 1143213100-143213350
  misc_feature: Hotspot regions tissue of origin
aatcatcgagtggaatcgaatggaattatgatcaaatggaatcgaatgtaatcatcatcaaatggaatcaaaaataaccatcatcaattggtattgaatggaattgtcatcaaatggaattcaaaggaatcatcatcaaatggaaccgaatggaatcctcattgaatggaaatgaaaggggtcatcatctaatggaatcgcatggaatcatcatcaaatggaatcgaatggaatcatcatcaaatggaat

SEQ ID NO: 35 - DNA, 650 bp, Homo sapiens
  misc_feature: 1143214250-143214900
  misc_feature: Hotspot regions tissue of origin
aaaatggaatcgaatggaatcaacatcaaatggaatcaaatggaatcattgaacggaattgaatggaatcgtcatcgaatgaattgaatgcaatcatcgaatggtctcgaatggaatcatcttcaaatggaatggaatggaatcatcgcatagaatcgaatggaattatcatcgaatggaatcgaatggaatcaacatcaaacggaaaaaaacggaattatcgaatggaatcgaagagaatcatcgaatggacccgaatggaatcatctaatggaatggaatggaataatccatggactcgaatgcaatcatcatcgaatggaatcgaatggaatcatcgaatggactcgaatggaataatctttgaacggaatcgaatggaatcatcatcggatggaaatgaatggaatcatcatcgaatggaatcgaatagaattatggaatgaaatccagtgtgatcatcatcgaatggacccgaatggaatcatcatccaacggaagctaatggaatcaacatcgaatgaatcaaatggaaacaccatcgaattgaaacgaatggaattaccatgaaattgaaatggatggactcatcatcgaatggattcgaatggaatcatcgaataaaattgattgaaatcatcatcgaatgg

SEQ ID NO: 36 - DNA, 350 bp, Homo sapiens
  misc_feature: 1143232850-143233200
  misc_feature: Hotspot regions tissue of origin
atggaatcgtcatagaatggaatcgaatggattcattgaatggaatcagatggaatcatcgaatggactggaatggaatcattgaatggactcgaaagggatcattattgaatggaattgaatggaatcatcgaatggtctcgattggaatcattatcaaatggaatcgaatggaatcaccgaatagaatcgaatggaacaatcatcgaatggactcaaatggaattatcctcaaatggaatcgaatggaattatcgaatgcaatcgaatggaattatcgaatgcaatcgaatagaatcatcgaatggactcgaatggaatcatcgaatggaatggaatggaacagtcaa

SEQ ID NO: 37 - DNA, 150 bp, Homo sapiens
  misc_feature: 1143264550-143264700
  misc_feature: Hotspot regions tissue of origin
catcgaatggactcgaatggaataatcattgaacggaatcgaatggaatcatcatcggatggaaacgaatggaatcatcatcgaatggaaatgaaaggagtcatcatctaatggaattgcatggaatcatcatcaaatggaatcgaatgg

SEQ ID NO: 38 - DNA, 50 bp, Homo sapiens
  misc_feature: 289831000-89831050
  misc_feature: Hotspot regions tissue of origin
ttccattccattccattccattccattccattccactccattccattccg

SEQ ID NO: 39 - DNA, 150 bp, Homo sapiens
  misc_feature: 289836450-89836600
  misc_feature: Hotspot regions tissue of origin
atggattggaaccaacccgagtggaatggaatggaatggaatggaatggaatggaatcaacccgagtgcaatggaatggaattgaatggaatggaatggaatcaaccctagtggaatggaaaggaatggaatggaatggaatggaacaga

SEQ ID NO: 40 - DNA, 100 bp, Homo sapiens
  misc_feature: 289841050-89841150
  misc_feature: Hotspot regions tissue of origin
aatgaaatggaatggaatgcaatagaatggaatggaatggaatggaatcaacacgaatggaatggaatggaatggaatggaatggaatggaatggaatgg

SEQ ID NO: 41 - DNA, 300 bp, Homo sapiens
  misc_feature: 375669100-75669400
  misc_feature: Hotspot regions tissue of origin
cattccggagcccagggtccagatttggtttcagaatgagaggtcacgccagctgaggcagcaccggcgggaatctcggccctggcccgggagacgcggcctgcaagaaggcaggcgaaagcggaccgccgtcaccggatcccagaccgccctgctcctccgagcctttgagaaggatcgctttccaggcatcgccgccagggaagagctggccagagagacgggcctcccggagtccaggattcagatctggtttcagaatcgaagggccaggcacccgggacagggtggcagggcgcc

SEQ ID NO: 42 - DNA, 450 bp, Homo sapiens
  misc_feature: 549658340-49658790
  misc_feature: Hotspot regions tissue of origin
tcgggttgattccattccattccattccattccattccattccattccattccattgcactggagttgattccattccattgcattccattccattccattgcattccattccattcctttccattccattccaatcgggttgattccgttctattcttttccattccattccattcccttccattccactcgggttgattccattcctttccattccactcgaaaacattcccttttattccactcggaatgattccactccattccattccattccattgaatttcgttccattccattctattccgtaccattccattccattccattccattccattccatgccattccactcgggttgattccattcctttccattacattcgtgttaaatccattccattccattccattccattccattccattccattcgattccattccat

SEQ ID NO: 43 - DNA, 250 bp, Homo sapiens
  misc_feature: 549659390-49659640
  misc_feature: Hotspot regions tissue of origin
tctattccgtaccattccactcctttccattccattccattccatgccattccactcgggttgattccgttccactccattccattcgggttgattccattccattccattccattccattccattccattccattccattccattgctctcgggttgattccattccattcgattccattccattccattccattccattccattccattccattccattcgtttccattccattccactcgtgttgat

SEQ ID NO: 44 - DNA, 300 bp, Homo sapiens
  misc_feature: 549661570-49661870
  misc_feature: Hotspot regions tissue of origin
tcggaatgattccactccattccattccattccattcaatttcgttccattccattctattccgtaccattccattccattccattccatggcattccattccattccattccattccatgccattacactcgggttgattccatgcctttccataacattcgggttgaatcctttccattccattccattccattcgattccattccattgcactcggtttgattccattccattccattccattccactccattccattccattccactccattccattccattccactccattccat

SEQ ID NO: 45 - DNA, 150 bp, Homo sapiens
  misc_feature: 6157310800-157310950
  misc_feature: Hotspot regions tissue of origin
atagggtcccagtgtgcactcacccggcctcatcatagtgtcccagtgtgcactcacctagcctcatcatagtgccccagtgtgcactcacctagcctcatcatagtgccccagtgtgcactcacctagcctcatcatagtgccccagtg

SEQ ID NO: 46 - DNA, 50 bp, Homo sapiens
  misc_feature: 6157314200-157314250
  misc_feature: Hotspot regions tissue of origin
cacctggcctcatcatagtgccccagtgtgcactcacctggcctcatcat

SEQ ID NO: 47 - DNA, 100 bp, Homo sapiens
  misc_feature: 759995750-59995850
  misc_feature: Hotspot regions tissue of origin
atcattctcagcaaattctttgtgatgtgtgcgttcaactcacatagtttaacctttcttttcatagagcagtttggaaacactctgtttgtaaagtctg

SEQ ID NO: 48 - DNA, 150 bp, Homo sapiens
  misc_feature: 843237700-43237850
  misc_feature: Hotspot regions tissue of origin
gttcagtatataatatgtatttggtgtactttttgatattttatgtacagtatataatatgtattttgggtactttgatattttatgtacagtatataataagtatttggtgtactttgatattttatgtacagtatataacatgtattt

SEQ ID NO: 49 - DNA, 150 bp, Homo sapiens
  misc_feature: 942900300-42900450
  misc_feature: Hotspot regions tissue of origin
aaaacaaacaaacaaacaaaaaacaaccagaaaaggatagggctgaagaacagaggtcgctgcatttagaaaggaggcggggtcagaggaatagaaagggatagggctgaagaacagaggtcactgcatttagaaaggaagtggggtcag

SEQ ID NO: 50 - DNA, 150 bp, Homo sapiens
  misc_feature: 1041859850-41860000
  misc_feature: Hotspot regions tissue of origin
gaatggtatggactggaatgtaatggaatggaatcaatccgagtggaatgtaatggaatggaatggaatggaatggaatcaacccgagtgcaatggaatggaatggaatggaatggaatggaatggaatagaatggaatcaattcgagtg

SEQ ID NO: 51 - DNA, 700 bp, Homo sapiens
  misc_feature: 10133688150-133688850
  misc_feature: Hotspot regions tissue of origin
agtgtagagatatgtcacaatgtcccctgtaggcagagcacagagaagagttgcaccacctgggtgatcagtgcagagatatgtcacaatgtcccctgtaggcaaagcctaggcaagagttacatcacctttgtcatcagttcagggatatgtgaaaacgcccctgtaggcagagcttagacaagagttacatcacctagttgatcagtgcagagatatttcacaataccccctgtaggcagatcctagacaagagttgcatcacctgggtgatcagtgcagagatatttcacaacgccccctgtaggcagagggtagacaagagttacatcacctaggtgatcagtgcagagatttgtcgaaattccctgtaggcagtgcttataaaagtgttacatcacctaagtgatcagtgcagagatatgtcacaaagctcctgtaggcagagcttagatgagttacatcacctgggtgatcagagcaaaggtatgtcacaaagccccctgtaggccaaagcctagacaatagttacatcacttgggtgatcagtggcgagatctctcacaattcccctgtaggcagagcttatacaacagttacatcacctgggtgatcagtgcagatatatgtcacaatgcccccataggcagatccaagacaagagtccgtctcctgggtgatcagtgcagaaatacgtcac

SEQ ID NO: 52 - DNA, 50 bp, Homo sapiens
  misc_feature: 10133689200-133689250
  misc_feature: Hotspot regions tissue of origin
gtaggcagagcctagacaagagttacatgacctaggtgatcagtgcagag

SEQ ID NO: 53 - DNA, 150 bp, Homo sapiens
  misc_feature: 10133689350-133689500
  misc_feature: Hotspot regions tissue of origin
agagaagagttatatcacctgggtgatcagtgcagagatatgtcacaagccccctgtaggcagagcctagacaagagttatatcacctggttgatcagtgcagtgatatgtcacaatgctgtgtagccagagcctaggcaaaagttacag

SEQ ID NO: 54 - DNA, 150 bp, Homo sapiens
  misc_feature: 10133689750-133689900
  misc_feature: Hotspot regions tissue of origin
agagcctagacaagagttacatcacctgggtgatcagtgcagagatatgtgacaaggcccctttaagcagagcctagacaatagttacatcacctgagtgatcagtgcagagatctgtcacaatgcccctttaggcagagcttagactag

SEQ ID NO: 55 - DNA, 50 bp, Homo sapiens
  misc_feature: 1517076150-17076200
  misc_feature: Hotspot regions tissue of origin
ccaaagaaagtttcaactatgtgagatgaatgcacacatcacaaagaagt

SEQ ID NO: 56 - DNA, 50 bp, Homo sapiens
  misc_feature: 1634588170-34588220
  misc_feature: Hotspot regions tissue of origin
tggactcgaatggaatcatcttcgagtagaatctaaaggattcactaagt

SEQ ID NO: 57 - DNA, 150 bp, Homo sapiens
  misc_feature: 1646387830-46387980
  misc_feature: Hotspot regions tissue of origin
catcgattggaatcgaatggaatcattgaatggactccaatggaatcatcaccaaatataatcaaaagaaatcatcaaatggattcgaatagaatcatcgaatggactcgaatggaatcatcatcgaatggaatcaaatggaatcatcaa

SEQ ID NO: 58 - DNA, 350 bp, Homo sapiens
  misc_feature: 1646389830-46390180
  misc_feature: Hotspot regions tissue of origin
acttgaatgaaatcacctttgaatggaatcaaagggaatgatcgaaaggaatcgaatgccatcatcatcgaatggaatcgaaaggaatcatcgaatggaattgaatggaatcaccattgaatggactcgaatggaatcatcatcgtatggaatcgaatggaatcatcatcatatggaatcaaatggaatcatcgaatggacaagaatggaatcatcgtcaaatggaatcaagtggatcatcgaatagaaactaatggaatcattgttgaaagaaatggaatggaatcattgaatggaattgagtggaatcaccaatgaatggaatcgaatggaatcatcatcaaatggaa

SEQ ID NO: 59 - DNA, 100 bp, Homo sapiens
  misc_feature: 1646399420-46399520
  misc_feature: Hotspot regions tissue of origin
tcaaacggaatcattgaatggaatcgaatggaatcaatgtcaagtggaatcgagtggaatcatcgaaagaaatcgaatggaatcattgtcgaatggaatg

SEQ ID NO: 60 - DNA, 200 bp, Homo sapiens
  misc_feature: 1726884050-26884250
  misc_feature: Hotspot regions tissue of origin
acagaaaagtttctgagaaggcttctgtctagatgttatgtgaagatatacccgtttcgaacgaaggccacagagtggtccaaatatccacttgtaggtcctggaaaaagagggtttcaaaactgaactttccaaggaaggtgcaactctgggatttgaatgcaaacatcacaaagaagattctgagactgcttctgttt

SEQ ID NO: 61 - DNA, 100 bp, Homo sapiens
  misc_feature: 18108400-108500
  misc_feature: Hotspot regions tissue of origin
ttgtgacatatctctgcactgatcacccaggtgatataactcttctctaggatctgcctacagggtgctttgtgacatatccctgcaatgatcacccagg

SEQ ID NO: 62 - DNA, 100 bp, Homo sapiens
  misc_feature: 1926161100-26161200
  misc_feature: Hotspot regions tissue of origin
gacagaatgattctcagaaactcctttgtgatgtgtgtgttcaactcacagagtttaacatttcttttcatagagcagttaggaaacactctgtttgtaa

SEQ ID NO: 63 - DNA, 100 bp, Homo sapiens
  misc_feature: 217926350-7926450
  misc_feature: Hotspot regions tissue of origin
gtgtccattccattcgtgtccactgcattccattccactccattccatattattccattacactccattctattctgttactttcatttccattcaattc

SEQ ID NO: 64 - DNA, 350 bp, Homo sapiens
  misc_feature: 2218896200-18896550
  misc_feature: Hotspot regions tissue of origin
ctgcctcacgggttcaagcgattgtcgtgcctcatcctccctggtcgctgggactacaccacactgggactacaccagctgccaccatgcctggctaatttttcgtatttttagtagagacagggtttcactgtattggccaggatggtcttgatctcctccccttttgatcctcttgccttggcctcccaaagtgctgggattacaggcctgagccaagatacatattttttaaaggaagaaaaatttcaaaggtactctgcttggtacaataatcaaatatataaattgaggaataaaacataaccatgaaacatatttataactgcctatggaaaatacagaggata

SEQ ID NO: 65 - DNA, 505 bp, Artificial
  Synthetic
  misc_feature: MT-ND5--MOB1A
  misc_feature: Mitochondrial predicted kinase druggable chimeras
accaccctcgttaaccctaacaaaaaaaactcatacccccattatgtaaaatccattgtcgcatccacctttattatcagtctcttccccacaacaatattcatgtgcctagaccaagaagttattatctcgaactgacactgagccaccgtgcccggcctatagaaatcaatctttttgactcttctcacttttatctccccatgcccaaggtttgcctgttccataacactcactcccttcccccttgctaatcagaagccatctcctctcagtgtctgatctctgctcttcatacatgattacagtcatgggggagaagagggcttgctaaattatgcagttaatcctatggcgcttaaattacaggccttaaaacaacactggtccagtgtggcgcatttttaaaaacaggagatcttctttgcccacattttttgtagacacaccaggtttggtcttatattttacagatggaagaaaaagtgatttttacagggcttcc

SEQ ID NO: 66 - DNA, 2675 bp, Artificial
  Synthetic
  misc_feature: AKAP12--ND1
  misc_feature: Mitochondrial predicted kinase druggable chimeras
acctcgcgggcgcgcgtcttttggctcttgcccctgtccctgcggcttggggaaggcgtaacccggcggctaggcgcgggagaagtgcggaggagccatgggcgccgggagctccaccgagcagcgcagcccggagcagccgcccgaggggagctccacgccggctgagcccgagcccagcggcggcggcccctcggccgaggcggcgccagacaccaccgcggaccccgccatcgctgcctcggaccccgccaccaagctcctacagaagaatggtcagctgtccaccatcaatggcgtagctgagcaagatgagctcagcctccaggagggtgacctaaatggccagaaaggagccctgaacggtcaaggagccctaaacagccaggaggaagaagaagtcattgtcacggaggttggacagagagactctgaagatgtgagcgaaagagactccgataaagagatggctactaagtcagcggttgttcacgacatcacagatgatgggcaggaggagacacccgaaataatcgaacagattccttcttcagaaagcaatttagaagagctaacacaacccactgagtcccaggctaatgatattggatttaagaaggtgtttaagtttgttggctttaaattcactgtgaaaaaggataagacagagaagcctgacactgtccagctactcactgtgaagaaagatgaaggggagggagcagcaggggctggcgaccaccaggaccccagccttggggctggagaagcagcatccaaagaaagcgaacccaaacaatctacagagaaacccgaagagaccctgaagcgtgagcaaagccacgcagaaatttctcccccagccgaatctggccaagcagtggaggaatgcaaagaggaaggagaagagaaacaagaaaaagaacctagcaagtctgcagaatctccgactagtcccgtgaccagtgaaacaggatcaaccttcaaaaaattcttcactcaaggttgggccggctggcgcaaaaagaccagtttcaggaagccgaaggaggatgaagtggaagcttcagagaagaaaaaggaacaagagccagaaaaagtagacacagaagaagacggaaaggcagaggttgcctccgagaaactgaccgcctccgagcaagcccacccacaggagccggcagaaagtgcccacgagccccggttatcagctgaatatgagaaagttgagctgccctcagaggagcaagtcagtggctcgcagggaccttctgaagagaaacctgctccgttggcgacagaagtgtttgatgagaaaatagaagtccaccaagaagaggttgtggccgaagtccacgtcagcaccgtggaggagagaaccgaagagcagaaaacggaggtggaagaaacagcagggtctgtgccagctgaagaattggttgaaatggatgcagaacctcaggaagccgaacctgccaaggagctggtgaagctcaaagaaacgtgtgtttccggagaggaccctacacagggagctgacctcagtcctgatgagaaggtgctgtccaaaccccccgaaggcgttgtgagtgaggtggaaatgctgtcatcacaggagagaatgaaggtgcagggaagtccactaaagaagctttttaccagcactggcttaaaaaagctttctggaaagaaacagaaagggaaaagaggaggaggagacgaggaatcaggggagcacactcaggttccagccgattctccggacagccaggaggagcaaaagggcgagagctctgcctcatcccctgaggagcccgaggagatcacgtgtctggaaaagggcttagccgaggtgcagcaggatggggaagctgaagaaggagctacttccgatggagagaaaaaaagagaaggtgtcactccctgggcatcattcaaaaagatggtgacgcccaagaagcgtgttagacggccttcggaaagtgataaagaagatgagctggacaaggtcaagagcgctaccttgtcttccaccgagagcacagcctctgaaatgcaagaagaaatgaaagggagcgtggaagagccaaagccggaagaaccaaagcgcaaggtggatacctcagtatcttgggaagctttaatttgtgtgggatcatccaagaaaagagcaaggagagggtcctcttctgatgaggaagggggaccaaaagcaatgggaggagaccaccagaaagctgatgaggccggaaaagacaaagagacggggacagacgggatccttgctggttcccaagaacatgatccagggcagggaagttcctccccggagcaagctggaagccctaccgaaggggagggcgtttccacctgggagtccgaactagtctcaggcttcaacatcgaatacgccgcaggccccttcgccctattcttcatagccgaatacacaaacattattataataaacaccctcaccactacaatcttcctaggaacaacatatgacgcactctcccctgaactctacacaacatattttgtcaccaagaccctacttctaacctccctgttcttatgaattcgaacagcatacccccgattccgctacgaccaactcatacacctcctatgaaaaaacttcctaccactcaccctagcattacttatatgat

SEQ ID NO: 67 - DNA, 1060 bp, Artificial
  Synthetic
  misc_feature: ND1--RACK1
  misc_feature: Mitochondrial predicted kinase druggable chimeras
atataagtaatgctagggtgagtggtaggaagttttttcataggaggtgtatgagttggtcgtagcggaatcgggggtatgctgttcgaattcataagaacagggaggttagaagtagggtcttggtgacccttcgtggcaccctcaagggccacaacggctgggtaacccagatcgctactaccccgcagttcccggacatgatcctctccgcctctcgagataagaccatcatcatgtggaaactgaccagggatgagaccaactatggaattccacagcgtgctctgcggggtcactcccactttgttagtgatgtggttatctcctcagatggccagtttgccctctcaggctcctgggatggaaccctgcgcctctgggatctcacaacgggcaccaccacgaggcgatttgtgggccataccaaggatgtgctgagtgtggccttctcctctgacaaccggcagattgtctctggatctcgagataaaaccatcaagctatggaataccctgggtgtgtgcaaatacactgtccaggatgagagccactcagagtgggtgtcttgtgtccgcttctcgcccaacagcagcaaccctatcatcgtctcctgtggctgggacaagctggtcaaggtatggaacctggctaactgcaagctgaagaccaaccacattggccacacaggctatctgaacacggtgactgtctctccagatggatccctctgtgcttctggaggcaaggatggccaggccatgttatgggatctcaacgaaggcaaacacctttacacgctagatggtggggacatcatcaacgccctgtgcttcagccctaaccgctactggctgtgtgctgccacaggccccagcatcaagatctgggatttagagggaaagatcattgtagatgaactgaagcaagaagttatcagtaccagcagcaaggcagaaccaccccagtgcacctccctggcctggtctgctgatggccagactttgtttgctggctacacggacaacctggtgcgagtgtggcaggtgaccattggcac

SEQ ID NO: 68 - DNA, 643 bp, Artificial
  Synthetic
  misc_feature: ATP8--MAPK3
  misc_feature: Mitochondrial predicted kinase druggable chimeras
accgtatggcccaccataattacccccatattccttacactattcctcaagtacatccactccgccaacgtgctccaccgagatctaaagccctccaacctgctcatcaacaccacctgcgaccttaagatttgtgatttcggcctggcccggattgccgatcctgagcatgaccacaccggcttcctgacggagtatgtggctacgcgctggtaccgggccccagagatcatgctgaactccaagggctataccaagtccatcgacatctggtctgtgggctgcattctggctgagatgctctctaaccggcccatcttccctggcaagcactacctggatcagctcaaccacattctgggcatcctgggctccccatcccaggaggacctgaattgtatcatcaacatgaaggcccgaaactacctacagtctctgccctccaagaccaaggtggcttgggccaagcttttccccaagtcagactccactgcccttgacctgctggaccggatgttaacctttaaccccaataaacggatcacagtggaggaagcgctggctcacccctacctggagcagtactatgacccgacggatgagccagtggccgaggagcccttcaccttcgccatggagctgg

SEQ ID NO: 69 - DNA, 523 bp, Artificial
  Synthetic
  misc_feature: MT-RNR1--STRAP
  misc_feature: Mitochondrial predicted kinase druggable chimeras
agcatccccgttccagtgagttcaccctctaaatcaccacgatcaaaagggacaagcatcaagcacgcagcaatgcagctcaaaacgcttagcctagccacacccccacgggaaacagcagtgattaacctttagcaataaacgaaagtttaactaagctatactaaccccagggttggtcaatttcgtgccagccaccgcggtcacacgattaacccaagtcaatagaagccggcgtaaagagtgttttagatcaccccctccccaataaagctaaaactcacctgagttgtaaaaaactcctgccgatgccggtgtggacgctgtgaatcgtggctggcccggttctccgcttctccccatcccctactttcctccctccctccctttccctccctcgtcgactgttgcttgctggtcgcatactccctgacccctccctcacccctccctaacctcggtgccaccggattgcccttcttttcctgttgcccagcccagccctagtgtcagggcgggggcc

SEQ ID NO: 70 - DNA, 608 bp, Artificial
  Synthetic
  misc_feature: TESK1--MT-ND2
  misc_feature: Mitochondrial predicted kinase druggable chimeras
acttcgggctggccgaaaagattcctgtgtatagggagggggcaaggaaggagccatggccgtggtgggctccccatactggatggctccagaggtgttacggggtgagctgtatgatgagaaggctgatgtctttgccttcgggattgtcctctgtgagctcatcgcccgagtacctgcagaccctgactacctaccacgcactgaggactttggcctggatgtgcctgctttccgaactctggtgggggatgactgcccactgcctttcttgctcctggccatccactgctgcaacctggaacccagcacccgtgcccccttcaccgaaattacccagcacctggaatggatcctggagcagctgcctgagccagccccactcaccaggaccgccctgacacacaatcagggctctgttgcaagagggggtccctctgccacgcttcccaggccagatccccggctttcccgaagccggtcagacctcttcctgcccccactaaccggctttttgcccaaatgggccattatcgaagaattcacaaaaaacaatagcctcatcatccccacatcatagccaccatcaacctccttaactctattct

SEQ ID NO: 71 - DNA, 520 bp, Artificial
  Synthetic
  misc_feature: MT-RNR1--RACK1
  misc_feature: Mitochondrial predicted kinase druggable chimeras
agcctagccacacccccacgggaaacagcagtgattaacctttagcaataaacgaaagtttaactaagctatactaaccccagggttggtcaatttcgtgccagccaccgcggtcacacgattaacccaagtcaatagaagccggcgtaaagagtgttttagatcaccccctccccaataaagctaaaactcacctgagttgtaaaaaactccagttgacacaaaatagactacgaaagtggctttaacatactttcactgcaaggcggcggcaggagaggttgtggtgctagtttctctaagccatccagtgccatcctcgtcgctgcagcgacacacgctctcgccgccgccatgactgagcagatgacccttcgtggcaccctcaagggccacaacggctgggtaacccagatcgctactaccccgcagttcccggacatgatcctctccgcctctcgagataagaccatcatcatgtggaaactgaccagggatgagaccaactatggaattccac

SEQ ID NO: 72 - DNA, 501 bp, Artificial
  Synthetic
  misc_feature: MT-RNR2--CAMK4
  misc_feature: Mitochondrial predicted kinase druggable chimeras
agtccaaagaggaacagctctttggacactaggaaaaaaccttgtagagagagtaaaaaatttaacacccatagtaggcctaaaagcagccaccaattaagaaagcgttcaagctcaacacccactacctaaaaaatcccaaacatataactgaactcctcacacccaattggaccaatctatcaccctatagaagaactaatgttagtataagtaacatgaaaacattctcctccgcataagcctgcgtcagattaaaacactctctcgctcctgcgttcgcaggcggcggctggcggccggcttctcgctcgggcagcggcggcggcggcggcggcggcttccggagtcccgctgcgaagatgctcaaagtcacggtgccctcctgctccgcctcgtcctgctcttcggtcaccgccagtgcggccccggggaccgcgagcctcgtcccggattactggatcgacggctccaacagggatgcgctgagcgatttcttcg

SEQ ID NO: 73 - DNA, 110 bp, Artificial
  Synthetic
  misc_feature: CYTB--CDK19
  misc_feature: Mitochondrial predicted kinase druggable chimeras
aaccccctaataaaattaattaaccactcattcatcgacctccccaccccatctctactaaaaaaaaaaaaaaaatacaaaactacaaaaaattagctgggcgtggtgtt

SEQ ID NO: 74 - DNA, 96 bp, Artificial
  Synthetic
  misc_feature: ATP8--ATP6
  misc_feature: Mitochondrial predicted chimeras
ataacaaaccctgagaaccaaatgaacgaaaatctgttcgcttcattcattgccccctctattgatccccacctccaaatatctcatcaacaaccg

SEQ ID NO: 75 - DNA, 98 bp, Artificial
  Synthetic
  misc_feature: RNR2--ATP8
  misc_feature: Mitochondrial predicted chimeras
agccaccaattaagaaagcgttcaagctcaacacccactacctaaaaatgcccaactaaatactaccgtatggccaccataattaccccatactcctt

SEQ ID NO: 76 - DNA, 251 bp, Artificial
  Synthetic
  misc_feature: ND1--ND1
  misc_feature: Mitochondrial predicted chimeras
actagcagagaccaactgaacccccttcgaccttgccgaagggagtccgaactagtctcaggcttcaacatcgaataccccaccccttcgccctattcttcatagccgaatacacaaacattattataataaacaccctcaccactacaatcttcctaggaacaaaatatgttgtgtagagttcaggggagagtgcgtcatatgttgttcctaggaagattgtagtggtgagggtgtttattataataatg

SEQ ID NO: 77 - DNA, 591 bp, Artificial
  Synthetic
  misc_feature: ND4--CLSPN
  misc_feature: Mitochondrial predicted chimeras
atgatgtcggggttgagggataggaggagaatgggggataggtgtatgaacatgagggtgttttctcgtgtgaatgagggttttatgttgttaatgtggtgggtgagtgagccgagattgcgccactgcactccagactgggcaacaaagtgagactatctcaaaaaaaaaaaaagaaatctttctttcaccgagccctggtcatcatggtacgtatctcatgggtgtaaaggagtcattatggtactgattttcactgtttaatctgtaatgtcacacttttccctactcttcctcttccaaccacttctatcaatcacaaagagatattaacccagaaagctgctacaataaaatgtcaaatgtctaatggaggaagtgaaacctaacccaaggccaggtgcagtgactcatgcatgtgaccagcactttgtggggccgaggtggatggatcatttgaggtcaggagttcaagaccagcctggccaacatggtgaaaccccgtctctactaaaaatacaaaaattagctgggcgtattggcacatgcctgtaatcccagctactcgggaggctgaaggaggagaatcgc

SEQ ID NO: 78 - DNA, 573 bp, Artificial
  Synthetic
  misc_feature: EWSR1/ERG
  misc_feature: Predicted druggable genomic fusions
agttacccaccccaaactggatcctacagccaagctccaagtcaatatagccaacagagcagcagctacgggcagcagagcagtggccagatccagctttggcagttcctcctggagctcctgtcggacagctccaactccagctgcatcacctgggaaggcaccaacggggagttcaagatgacggatcccgacgaggtggcccggcgctggggagagcggaagagcaaacccaacatgaactacgataagctcagccgcgccctccgttactactatgacaagaacatcatgaccaaggtccatgggaagcgctacgcctacaagttcgacttccacgggatcgcccaggccctccagccccaccccccggagtcatctctgtacaagtacccctcagacctcccgtacatgggctcctatcacgcccacccacagaagatgaactttgtggcgccccaccctccagccctccccgtgacatcttccagtttttttgctgccccaaacccatactggaattcaccaactgggggtatataccccaacactaggctccccaccagccatatg

SEQ ID NO: 79 - DNA, 405 bp, Artificial
  Synthetic
  misc_feature: BCR/ABL1
  misc_feature: Predicted druggable genomic fusions
aagcctgtggaccgtgtgacgaggagcacgctggtcctccatgacttgctgaagcacactcctgccagccaccctgaccaccccttgctgcaggacgccctccgcatctcacagaacttcctgtccagcatcaatgaggagatcacaccccgacggcagtccatgacggtgaagaagggagagggagaagacaggatgaaagcttcatcaacgaggaagagattactccttatggaagaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcata

SEQ ID NO: 80 - DNA, 425 bp, Artificial
  Synthetic
  misc_feature: LOC105369736/LRRK2
  misc_feature: Predicted druggable genomic fusions
ggtagttagataactggtatgtttaagcctggcacacaatgggtttcatatatatatatattttcaaagaatgaatgaatgagaatttatccttgaagaggccctacaaagattcattgatctgaggagaagtaaagagatagagccacagctgaggtaataccaggagcagggctggtaggcctgaataaatggttgacttattttataaatgtctcagctagatctgtattcagaaaaacaacaaatatctctgtgaccttccacaattccctttaatatatgcaaaccaatgaaaataggttaaaaatgaacaatctttgctgatgcaataacaattatgcacacaattcagtcgtttaaaagatctgtaatttccccattgatcaaattcactaccatacctttgcttggaaaaggaattg

SEQ ID NO: 81 - DNA, 335 bp, Artificial
  Synthetic
  misc_feature: JAKMIP1/FLI1
  misc_feature: Predicted druggable genomic fusions
agtagagaacttgagtaggttgagactatggtgtgtgtgtgtgtgtgtgtgtgtgtgtgtgtgtgtgtgtgtgtgtgtgtgtttgtgtgtgtgtgtgtttgcgagagagagagagagagagagagagagagagagagagagagagagagagagatcctgaataatgcacgaggtatttggactttgaaggcaacatacctttgaagatttctgggtagggatgtgacatgatgaaaatgatgtccagggtggactggttgctcaggatggccctagggatgacgggctgcctgtaataggaccacgagggaagcaaggagctttatcttgaggct

SEQ ID NO: 82 - DNA, 479 bp, Artificial
  Synthetic
  misc_feature: BTK/CYB561D1
  misc_feature: Predicted druggable genomic fusions
agattgtaatgttatagtccctgtactttctatttgaaatatttcataatataaaacaaagtatatttgaggccaggtgcagcagctcatgcctgtggtccccacactttggaaggctgagacaagaagatcacttgcatccaggagtttgagactagcctaggcaacaaagtgagacccgtgtctctacaaaaaaattaaaaattagctgggactacaggcacgtgccaccatatccagctaatttttgtatttttagtagagatggggtttcaccatgttggccaggatctcttgacctcgtgatctgcccgcctcggcctcccaaagtgctgtgattacaggcgtgagccaccgtgcctagccaggtcttgttttgaaaacctcactgtgggagattcaagcatcctccctaagccagctggccgctgtgctaaagcctgttcagagttaataataatcattagctgaatggtgct

SEQ ID NO: 83 - DNA, 702 bp, Artificial
  Synthetic
  misc_feature: SNAPC5/MAP2K1
  misc_feature: Predicted druggable genomic fusions
ggatgtcaactgctgagttctacatgaaaagcaaataataataataaaataacatgatgtacactctctgatgctcattttcatagcagcaaagcatgcttcacatgcactgcctgtgaaggatctcacaaggctccctcctagagagaaagtatgcccagcaagaataaagtaccaccaaatgtcaaagaatcccgaaaatacaggcagacagccggtcatggagcaggaacagcagtgcaattgtaagaatgactacccacattcaccaaaatttcactgtaattcagcctggcacaggttttgatcatcagcatacacacaaatagccccaagcacaaagccaatccacttaggggaacaataatagtaataagaataaagacagtaatattaaaaatgacaagagtagaatcttggcacatgctgtgttcttcatcctttgtcacaggtgaaatgcacatctgaacagagacaggcatgggaaggaggcccaaaagcgacatggcaaaccaccgggcaggggactcgctctttgttgcttcccaaacacttagacgccagcagcatgggttggtgtgctgggctggttaaggccgatggtggagcagagccaacctgcaaaatccacttcctcagcatcagatctcttgaaataaagcatgaaccatgagttgcttcaaatctgctctctctgcgggg

SEQ ID NO: 84 - DNA, 350 bp, Artificial
  Synthetic
  misc_feature: LOC105369782/ ERBB3
  misc_feature: Predicted druggable genomic fusions
aagagatgggggtcttactatgttggctaagctggtctcaaactccttgcctcaggtaatccttctgcctcagcctcccaagtgctgaggattaaggaatgaggcaccatgcccgccttccctttgtgtaaaaggagataatatgtatgtcacagaattgttttaaggatgataaaggactaagtgagataatgtaggtgaagtttagtatgatgcctggctcatagttaagtgctgaattatgatagtaatcaataactactcctaacctctttcttcacagattttaagcttctgtctttctagggggttaagagtcataaaccaaaattacactttcctctgctagg

SEQ ID NO: 85 - DNA, 478 bp, Artificial
  Synthetic
  misc_feature: BCR/PDGFRA
  misc_feature: Predicted druggable genomic fusions
acagagactggcagcgcaccgtcatcgccatgaatggggtacgtgtccgtgggactctcctggtgcccacttcccccagaaggatagggtggcctctgttcatttcaaatcagtcagaggtggctgagcctgaggcagcatctgagagggagcctggttggagaagggagggcccccaagagcagaatcaccatgcacgggaatcgtcattcattggctggaatgcagttgccagccaggccctgagcatccctcctcaaacaaaggtctcatggcaccaccaggacaggtggggcctccactcagggacctgggggctgcccatagaaatggagacccctgatttgtctttaggtaccccagaaaggtttagaccttaaaagcaatgacacacccaaaaaggcccgggatggacatgaatatatttatgtggacccgatgcagctgccttatgactcaagatgggagtttccaagag

SEQ ID NO: 86 - DNA, 1529 bp, Artificial
  Synthetic
  misc_feature: RPL7A/NTRK1
  misc_feature: Druggable kinase DNA chimeras
agatgccgaaaggaaagaaggccaagggaaagaaggtggctccggccccagctgtcgtgaagaagcaggaggctaagaaagtggtgaatcccctgtttgagaaaaggcctaagaattttggcattgacactaacagcacatctggagacccggtggagaagaaggacgaaacaccttttggggtctcggtggctgtgggcctggccgtctttgcctgcctcttcctctctacgctgctccttgtgctcaacaaatgtggacggagaaacaagtttgggatcaaccgcccggctgtgctggctccagaggatgggctggccatgtccctgcatttcatgacattgggtggcagctccctgtcccccaccgagggcaaaggctctgggctccaaggccacatcatcgagaacccacaatacttcagtgatgcctgtgttcaccacatcaagcgccgggacatcgtgctcaagtgggagctgggggagggcgcctttgggaaggtcttccttgctgagtgccacaacctcctgcctgagcaggacaagatgctggtggctgtcaaggcactgaaggaggcgtccgagagtgctcggcaggacttccagcgtgaggctgagctgctcaccatgctgcagcaccagcacatcgtgcgcttcttcggcgtctgcaccgagggccgccccctgctcatggtctttgagtatatgcggcacggggacctcaaccgcttcctccgatcccatggacctgatgccaagctgctggctggtggggaggatgtggctccaggccccctgggtctggggcagctgctggctgtggctagccaggtcgctgcggggatggtgtacctggcgggtctgcattttgtgcaccgggacctggccacacgcaactgtctagtgggccagggactggtggtcaagattggtgattttggcatgagcagggatatctacagcaccgactattaccgtgtgggaggccgcaccatgctgcccattcgctggatgccgcccgagagcatcctgtaccgtaagttcaccaccgagagcgacgtgtggagcttcggcgtggtgctctgggagatcttcacctacggcaagcagccctggtaccagctctccaacacggaggcaatcgactgcatcacgcagggacgtgagttggagcggccacgtgcctgcccaccagaggtctacgccatcatgcggggctgctggcacggggagccccagcaacgccacagcatcaaggatgtgcacgcccggctgcaagccctggcccaggcacctcctgtctacctggatgtcctgggctagggggccggcccaggggctgggagtggttagccggaatactggggcctgccctcagcatcccccatagctcccagcagccccagggtgatctcaaagtatctaattcaccctcagcatgtgggaagggacaggtgggggctgggagtagaggatgttcctgcttctctaggcaaggtcccgtcatagcaattatatttattatccctt

SEQ ID NO: 87 - DNA, 1606 bp, Artificial
  Synthetic
  misc_feature: TPR/MET
  misc_feature: Druggable kinase DNA chimeras
acccgagagtgtcaaagcttgcggcttgagctagagaaactcaacaatcaactgaaggcactaactgagaaaaacaaagaacttgaaattgctcaggatcgcaatattgccattcagagccaatttacaagaacaaaggaagaattagaagctgagaaaagagacttaattagaaccaatgagagactatctcaagaacttgaatacttaacagatcagtttcctaattcatctcagaacggttcatgccgacaagtgcagtatcctctgacagacatgtcccccatcctaactagtggggactctgatatatccagtccattactgcaaaatactgtccacattgacctcagtgctctaaatccagagctggtccaggcagtgcagcatgtagtgattgggcccagtagcctgattgtgcatttcaatgaagtcataggaagagggcattttggttgtgtatatcatgggactttgttggacaatgatggcaagaaaattcactgtgctgtgaaatccttgaacagaatcactgacataggagaagtttcccaatttctgaccgagggaatcatcatgaaagattttagtcatcccaatgtcctctcgctcctgggaatctgcctgcgaagtgaagggtctccgctggtggtcctaccatacatgaaacatggagatcttcgaaatttcattcgaaatgagactcataatccaactgtaaaagatcttattggctttggtcttcaagtagccaaaggcatgaaatatcttgcaagcaaaaagtttgtccacagagacttggctgcaagaaactgtatgctggatgaaaaattcacagtcaaggttgctgattttggtcttgccagagacatgtatgataaagaatactatagtgtacacaacaaaacaggtgcaaagctgccagtgaagtggatggctttggaaagtctgcaaactcaaaagtttaccaccaagtcagatgtgtggtcctttggcgtgctcctctgggagctgatgacaagaggagccccaccttatcctgacgtaaacacctttgatataactgtttacttgttgcaagggagaagactcctacaacccgaatactgcccagaccccttatatgaagtaatgctaaaatgctggcaccctaaagccgaaatgcgcccatccttttctgaactggtgtcccggatatcagcgatcttctctactttcattggggagcactatgtccatgtgaacgctacttatgtgaacgtaaaatgtgtcgctccgtatccttctctgttgtcatcagaagataacgctgatgatgaggtggacacacgaccagcctccttctgggagacatcatagtgctagtactatgtcaaagcaacagtccacactttgtccaatggttttttcactgcctgacctttaaaaggccatcgatattctttgctcttgccaaaattgcactattataggacttgtattgttatttaaattactggattctaagaatttcttatctgacagagcatcagaacagaggcttggtcccacaggccacggaccaatggcctgcagccgtgacaacactcctgtcatattggagtcc

SEQ ID NO: 88 - DNA, 332 bp, Artificial
  Synthetic
  misc_feature: PVT1/AKT3
  misc_feature: Druggable kinase DNA chimeras
agatgtgccccgggacccccggcaccttccagtggatttccttgcggaaaggatgttggcggtccctgtgacctgtggagacacggccagatctgccctccagagaatatataaaaaactggaggccaagatacttccttttgaagacagatggctcattcataggatataaagagaaacctcaagatgtggatttaccttatcccctcaacaacttttcagtggcaagaatgccagttaatgaaaacagaacgaccaaagccaaacacatttataatcagatgtctccagtggactactgttatagagagaacatttcatgtagatactcc

SEQ ID NO: 89 - DNA, 3474 bp, Artificial
  Synthetic
  misc_feature: SRGAP3/RAF1
  misc_feature: Druggable kinase DNA chimeras
accagagctgtgtgattaccagggcttgagagaggactcgaaaagaaaaggagaaataaatcaaacagcctgtcagttcctttggccacgtcagaagatgtcatctcaaactaagttcaagaaagacaaagagatcattgctgaatatgaagcccaaataaaagagatccgcacgcagctggtggagcagttcaaatgtctggagcagcaatcagagtcgcgactgcagctgcttcaagacctccaggagtttttccgccggaaagctgagattgagctcgagtactcccgcagcctggagaagctggctgagcgcttctcctccaaaatccgcagctcccgggagcaccagttcaagaaggaccagtacctcctctcgcctgtgaactgttggtatctggttctgcatcagacccggcgggagagccgagaccatgccaccctcaatgacatcttcatgaacaatgtcatcgtccgcctctcccagatcagtgaggatgtcatcagactcttcaaaaagagcaaggagattggcctgcagatgcacgaggagctcctgaaggtgaccaatgagctctacacagtcatgaaaacctaccacatgtaccatgcagagagcatcagtgcggaaagcaagctgaaggaggctgagaagcaggaggagaagcagttcaataagtcaggagacctcagcatgaacctgctccggcacgaggaccggccccagcgccgcagctctgtgaagaagattgagaagatgaaggagaagaggcaggccaagtactctgagaacaagctgaaatgcacaaaggcccggaatgactacttgctcaatctggcagccaccaacgcagctataagcaaatactacatccatgatgtctctgatctgatcgattgctgtgatttgggcttccatgccagcctggcccgcaccttccggacctatctctcagctgaatacaacctggagacctctcgccacgaagggctggatgtcattgagaatgcagtggacaacctggattcccgaagtgacaagcacacagtcatggacatgtgcaatcaagtcttctgccctccactcaagttcgagttccagccccacatgggggatgaggtctgccaggtcagcgctcagcagcccgtccagacagaactgctcatgcgttatcaccagctgcagtccagactggccaccctcaagatagagaatgaggaggttaggaaaaccctggatgccaccatgcagacattacaggacatgctgactgtggaggactttgatgtctccgatgccttccaacacagtcgatcgacagagtccgtcaagtcggctgcctctgagacctacatgagcaagatcaacattgccaagaggagagccaaccagcaggaaacagaaatgttttattttacaaaatttaaagagtatgtgaatggcagtaacctcatcaccaagctgcaggccaagcacgatttactcaagcagaccctgggcgaaggggaaagagcagaatgcggcaccaccaggcccccctgtcttccccctaaaccacagaaaatgaggagacctaggcctctctcagtgtatagccataaactctttaacggcagtatggaagcattcattaagaggcctcgtggacagagagattcaagctattattgggaaatagaagccagtgaagtgatgctgtccactcggattgggtcaggctcttttggaactgtttataagggtaaatggcacggagatgttgcagtaaagatcctaaaggttgtcgacccaaccccagagcaattccaggccttcaggaatgaggtggctgttctgcgcaaaacacggcatgtgaacattctgcttttcatggggtacatgacaaaggacaacctggcaattgtgacccagtggtgcgagggcagcagcctctacaaacacctgcatgtccaggagaccaagtttcagatgttccagctaattgacattgcccggcagacggctcagggaatggactatttgcatgcaaagaacatcatccatagagacatgaaatccaacaatatatttctccatgaaggcttaacagtgaaaattggagattttggtttggcaacagtaaagtcacgctggagtggttctcagcaggttgaacaacctactggctctgtcctctggatggccccagaggtgatccgaatgcaggataacaacccattcagtttccagtcggatgtctactcctatggcatcgtattgtatgaactgatgacgggggagcttccttattctcacatcaacaaccgagatcagatcatcttcatggtgggccgaggatatgcctccccagatcttagtaagctatataagaactgccccaaagcaatgaagaggctggtagctgactgtgtgaagaaagtaaaggaagagaggcctctttttccccagatcctgtcttccattgagctgctccaacactctctaccgaagatcaaccggagcgcttccgagccatccttgcatcgggcagcccacactgaggatatcaatgcttgcacgctgaccacgtccccgaggctgcctgtcttctagttgactttgcacctgtcttcaggctgccaggggaggaggagaagccagcaggcaccacttttctgctccctttctccagaggcagaacacatgttttcagagaagctgctgctaaggaccttctagactgctcacagggccttaacttcatgttgccttcttttctatccctttgggccctgggagaaggaagccatttgcagtgctggtgtgtcctgctccctccccacattccccatgctcaaggcccagccttctgtagatgcgcaagtggatgttgatggtagtacaaaaagcaggggcccagccccagctgttggctacatgagtatttagaggaagtaaggtagcaggcagtccagccctgatgtggagacacatgggattttggaaatcagcttctggaggaatgcatgtcacaggcgggactttcttcagagagtggtgcagcgccagacattttgcacataaggcaccaaacagcccaggactgccgagactctggccgcccgaaggagcctgctttggtactatggaacttttcttaggggacacgtcctcctttcacagcttctaaggtgtccagtgcattgggatggttttccaggcaaggcactcggccaatccgcatctcagccctctcagggagcagtcttccatcatgctgaattttgtcttccaggagctgcccctatggggcggggccgcagggccagccttgtttctctaacaaacaaacaaacaaacagccttgtttctctagtcacatcatgtgtatacaaggaagccaggaatacaggttttcttgatgatttgggttttaattttgtttttattgcacctgacaaaatacagttatctgatggtccctcaattatgtt

SEQ ID NO: 90 - DNA, 384 bp, Artificial
  Synthetic
  misc_feature: ZDHHC1/ABL1
  misc_feature: Druggable kinase DNA chimeras
acaagcctgtggaccgtgtgacgaggagcacgctggtcctccatgacttgctgaagcacactcctgccagccaccctgaccaccccttgctgcaggacgccctccgcatctcacagaacttcctgtccagcatcaatgaggagatcacaccccgacggcagtccatgacggtgaagaagggagaggctggtctcgaactcctgacctcagaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaag

SEQ ID NO: 91 - DNA, 801 bp, Artificial
  Synthetic
  misc_feature: NTRK2/NTRK2
  misc_feature: Druggable kinase DNA chimeras
actctaatagccaagaatgagtatagtccccattgttcatgtgagtgggattatccagctggaggcagccgtggtactccgtgtgattggtaacatgtattttagtacagatgtatttggactcattcaatattgccccgttatagaaccactcacatgaacaatggggactacactctaatagccaagaatgagtatgggaaggatgagaaacagatttctgctcacttcatgggctggcctggaattgacgatggtgcaaacccaaattatcctgatgtaatttatgaagattatggaactgcagcgaatgacatcggggacaccacgaacagaagtaatgaaatcccttccacagacgtcactgataaaaccggtcgggaacatctctcggtctatgctgtggtggtgattgcgtctgtggtgggattttgccttttggtaatgctgtttctgcttaagttggcaagacactccaagtttggcatgaaaggttttgttttgtttcataagatcccactggatgggtagctgaaataaaggaaaagacagagaaaggggctgtggtgcttgttggttgatgctgccatgtaagctggactcctgggactgctgttggcttatcccgggaagtgctgcttatctggggttttctggtagatgtgggcggtgtttggaggctgtactatatgaagcctgcatatactgtgagctgtgattggggaacaccaatgcagaggtaactctcaggcagctaagcagcacctcaagaaaacatgttaaattaatgcttctcttctt

SEQ ID NO: 92 - DNA, 536 bp, Artificial
  Synthetic
  misc_feature: GOLPH3L/AKT3
  misc_feature: Druggable kinase DNA chimeras
attccttctctgcatcgaaggatcaggaagtttgtgctctctgcgtggctaagtttttcacctactaggacgggggtggggtggggagaacaggtgtccttctaaaatacagcacaagctacagcctgcgtccagccataacccaggagtaacatcagaaacaggtgagaatgaccactttaactcaccgggcccgtcgcactgaaataagcaagaactctgaaaagaagatggaaagtgaggaagacagtaattgggagaaaagtccagacaatgaagattctggagactctaaggatatccgccttactcttatggaagaagtattgcttctgggactaaaagataaagaggctgagtcatcactagagagtgggaagggcagcagcagcagagaatccaaaccctaaagctgatatcacaaagtaccatttctccaagttgggggctcagaggggagtcatcatgagcgatgttaccattgtgaaagaaggttgggttcagaagaggggagaatatataaaaaactggaggcc

SEQ ID NO: 93 - DNA, 554 bp, Artificial
  Synthetic
  misc_feature: RNA45SN2/KIT
  misc_feature: Druggable kinase DNA chimeras
gacgacctgcttctgggtcggggtttcgtacgtagcagagcagctccctcgctgcgatctattgaaagtcagccctcgacacaagggtttgtaattgagaagcagatttcagagagcaccaatcatatttactccaacttagcaaacatgcagccccaaccgacagaagcccgtggtagaccattctgtgcggatcaattctgtcggcagcaccgcttcctcctcccagcctctgcttgtgcacgacgatgtctgagcagaatcagtgtttgggtcacccctccaggaatgatctcttcttttggcttccatgatggttattttcttttctttcaacttgcatccaactccaggatagtgggcaccccactgcaatcctgtctttctgagcacactttagtggccgatgatttttgtcatcagccaccatcctattgcaaaggttccaactgtatatattcccaatagcaacgtagcttctaccatgaacagagaacattctgatttggaaaaagagagggaggtatggactgggggccagagtcctttccaag

SEQ ID NO: 94 - DNA, 572 bp, Artificial
  Synthetic
  misc_feature: HSPB6/AKT1
  misc_feature: Druggable kinase DNA chimeras
acgcggaggagcaggatggagatccctgtgcctgtgcagccgtcttggctgcgccgcgcctcggccccgttgcccggactttcggcgcccggacgcctctttgaccagcgcttcggcgaggggctgctggaggccgagctggctgcgctctgccccaccacgctcgccccctactacctgcgcgcacccagcgtggcgctgcccgtcgcccaggtgccgacggaccccggccacttttcggtgctgctagacgtgaagcacttctcgccggaggaaattgctgtcaaggtggtgggcgaacacgtggaggtgcacgcgcgccacgaggagcgcccggatgagcacggattcgtcgcgcgcgagttccaccgtcgctaccgcctgccgcctggcgtggatccggctgccgtgacgtccgcgctgtcccccgagggcgtcctgtccatccaggccgcaccagcgtcggcccaggccccaccgccagccgcttctatggcgctgagattgtgtcagccctggactacctgcactcggagaagaacgtggtgtaccgggacctcaagctggagaac

SEQ ID NO: 95 - DNA, 578 bp, Artificial
  Synthetic
  misc_feature: MET/FAM3C2
  misc_feature: Druggable kinase DNA chimeras
cagatgcggagccgagtggagggcgcgagccagatgcggggcgacagctgacttgctgagaggaggcggggaggcgcggagcgcgcgtgtggtccttgcgccgctgacttctccactggttcctgggcaccgaaaggagcatattaatgaaaagtgccataaactgaaaaaccaaacatgagggtagcaggtgctgcaaagttggtggtagctgtggcagtgtttttactgacattttatgttatttctcaagtatttgaaataaaaatggatgcaagtttaggaaatctatttgcaagatcagcattggacacagctgcacgctctacaaagcctcccagatataagtgtgggatctcaaaagcttgccctgagaagcattttgcttttaaaatggcaagtggagcagccaacgtggtgggacccaaaatctgcctggaagataatgttttaatgagtggtgttaagaataatgttggaagagggatcaatgttgccttggcaaatggaaaaacaggagaagtattagacactaaatattttgacatgtggggaggagatgtggcaccatttattga

SEQ ID NO: 96 - DNA, 160 bp, Artificial
  Synthetic
  misc_feature: CDKN2A/CDKN2A
  misc_feature: Druggable kinase DNA chimeras
tccgcggcatctatgcgggcatggttactgcctctggtgccccccgcagccgcgcgcaggtaccgtgcgacatcgcgatggcccagctcctcagccaggtccacggccgcggcccggggtcgggtagaggaggtgcgggcgctgctggaggcgggggcgc

SEQ ID NO: 97 - DNA, 310 bp, Artificial
  Synthetic
  misc_feature: CDK4/ARV1
  misc_feature: Druggable kinase DNA chimeras
agcacagctgctgctggaaatgctgacttttaacccacacaagcgaatctctgcctttcgagctctgcagcactcttatctacataaggatgaaggtaatccggagtgagcaatggagtggctgccatggaaggaagaaaagctgccatttcccttctggacactgagagggcaatctttgcctttatctctgaggctatggagggtcctcctccatctttctacagagaaggaggaaagaatgatgaagaccgaagacgaactgacgtgaagagggaagcttaaggaacttgtgaaggtctcaattctc

SEQ ID NO: 98 - DNA, 612 bp, Artificial
  Synthetic
  misc_feature: CDK4/JHY
  misc_feature: Druggable kinase DNA chimeras
tgtttcttcctctgtttggctttgccattgtgcgatttggaaaaaccacttggaagaagggactttcctgcaaaaccttaaagactggttaaattacagggcctaggaagtcagtggagccccttgactgacaaagcttagaaaggaactgaaattgcttctttgaatatggattttaggcggggcgtggtggctcacgcctataatcccaccagacgtaattctttaagacaaaaaagggagtctctgaagaaaagccacaaatgttcatccacctcatctattttgttccattagaaaatgcttagaggttagaacaggttagaatatgttaaaaaaaaataccagttgggatttggaataggtctaagggcaaccacatttaacataataacctctcctttgtatttgaaaatgacctcacattagcaggtgataaatctcaagctctttccagtaagagtggtggactctaccattctaaccccactcttgtcctgtatccctggacatgtccataggaggtgggcaggaaggtctttatgtgcacctgtgaaatggaactaagtttactggctccaaaaggttttgaatccatcacgtgggagagaa

SEQ ID NO: 99 - DNA, 295 bp, Artificial
  Synthetic
  misc_feature: CDK12/CDK12
  misc_feature: Druggable kinase DNA chimeras
agtaacaggaaatatttaattttccaatctccactttccatttttataaactgaaagaaaaaaatttaatatattacaaaatatctgtacatagacatcacccaaccacttgtatgtgtgtgtgtatatataatatgcatatatagttaccgtgctaaaatggttaccagcaggttttgagagagaatgctgcatcagaaaagtgtcagttgccacctcattctccctgatttaggttcctgacactgattcctttctctctcgtttttgacccccattgggtgtatcttgcctc

SEQ ID NO: 100 - DNA, 631 bp, Artificial
  Synthetic
  misc_feature: MAP2K1/SLC48A1
  misc_feature: Druggable kinase DNA chimeras
ttgtgactgtgctacttgttgtgtaaccttggatgagtcatttaacttctccaaacctcaggaaaatggagaaaacagaacctacctaaagggttgtatgcagattaattaatacatacaaagcacttagaatagtacttaacatgtagtaggcacttaatacattttacctgttgcttagataaattggtagaggtttttatgattaccagcaacagaaaccagcctgacttggtttaagcttctaaaaatttattcattagaaggatctggctgggattataggtgtgagccaccgtgcccggcctggatctgttttcttagcacgcagtgaggaatctttgtacttaaggccaggcaacaaagtcaagaggtcaaggtgtagggccatgaggcctggacctatgctgcaggcaagggtttccatccccgctgccctaggcactctcttcccaaggccaggttgggcacctggggaggtcagttcagaaatatctagcagagacctcttaaacccccatcccagcaccccatcctgttgttcccagagctggtctcccatgagtgtgctagagccagatagccgtgggcccccaaccatctcactcacacacacaggcatccaccccca

SEQ ID NO: 101 - DNA, 444 bp, Artificial
  Synthetic
  misc_feature: IGH/CDK12
  misc_feature: Druggable kinase DNA chimeras
tatcacaatatttaccaattggcacaaagatgaattattcagtgcatattgttaaaaggaaaacaacaacaaaaacaagataacttattaatggtacaaaaaggggttaaattgtgcttccaggcaagaactttgccttatcataaacaggaaatgaaaaagggaagggctgtcaggatgggataatttgggaggcttctcattctggcttctatttctatgtgagtaccagcatatagagtgttttaaaaacagatacatgtcatataatttatctgcacagacttagaccttcaggaaacataggttaagcccccttttacaaagaaaaagtaaacatacttcagcatcttggagggtagttttcaaaactcaagtttcatgtttcaatgccaagttcttattttaaaaaataaaatctacttatagagaaaggtgcattac

SEQ ID NO: 102 - DNA, 248 bp, Artificial
  Synthetic
  misc_feature: AKT1/PPP3R1
  misc_feature: Druggable kinase DNA chimeras
cgcggcggcccgaggctgtggccaggccagctgggctcggggagcgccagcctgagaggagcgcgtgagcgtcgcgggagcctcgggcaccatgagcgacgtggctattgtgaaggagggttggctgcacaaacgaggggatggtttatgttgaatgtttagtttttcttctgcatgatacgtcatgttgtgggatctttagaaaacttcatactgtatgaataagaaaataaaatatttgaaacttg

SEQ ID NO: 103 - DNA, 2163 bp, Artificial
  Synthetic
  misc_feature: CDK4/RPL4P4
  misc_feature: Druggable kinase DNA chimeras
aggcggaccgagctcgggtgtatggggccgtaggaaccggctccggggccccgataacgggccgcccccacagcaccccgggctggcgtgagggtctcccttgatctgagaatggctacctctcgatatgagccagtggctgaaattggtgtcggtgcctatgggacagtgtacaaggcccgtgatccccacagtggccactttgtggccctcaagagtgtgagagtccccaatggaggaggaggtggaggaggccttcccatcagcacagttcgtgaggtggctttactgaggcgactggaggcttttgagcatcccaatgttgtccggctgatggacgtctgtgccacatcccgaactgaccgggagatcaaggtaaccctggtgtttgagcatgtagaccaggacctaaggacatatctggacaaggcacccccaccaggcttgccagccgaaacgatcaaggatctgatgcgccagtttctaagaggcctagatttccttcatgccaattgcatcgttcaccgagatctgaagccagagaacattctggtgacaagtggtggaacagtcaagctggctgactttggcctggccagaatctacagctaccagatggcacttacacccgtggttgttacactctggtaccgagctcccgaagttcttctgcagtccacatatgcccttaggtcctggtctacatgctcaaacaccagggttaccttgatctcccggtcagttcgggatgtggcacagacgtccacgggctgagaggagcgtggctgtctcctctctccgccatggcgtgtgctcgcccactgatatcggtgtactccgaaaagggggagtcatctggcaaaaatgtcactttgcctgctgtattcaaggctcctattcgaccagatattgtgaactttgttcacaccaacttgcgcaaaaacaacagacagccctatgctgtcagtgaattagcaggtcatcagactagtgctgagtcttggggtactggcagagctgtggctcgaattcccagagttcgaggtggtgggactcaccgctctggccagggtgcttttggaaacatgtgtcgtggaggccgaatgtttgcaccaaccaaaacctggcgccgttggcatcgtagagtgaacacaacccaaaaacgatacgccatctgttctgccctggctgcctcagccctaccagcactggtcatgtctaaaggtcatcgtattgaggaagttcctgaacttcctttggtagttgaagataaagttgaaggctacaagaagaccaaggaagctgttttgctccttaagaaacttaaagcctggaatgatatcaaaaaggtctatgcctctcagcgaatgagagctggcaaaggcaaaatgagaaaccgtcgccgtatccagcgcaggggcccgtgcatcatctataatgaggataatggtatcatcaaggccttcagaaacatccctggaattactctgcttaatgtaagcaagctgaacattttgaagcttgctcctggtgggcatgtgggacgtttctgcatttggactgaaagtgctttccggaagttagatgaattgtacggcacttggcgtaaagccgcttccctcaagagtaactacaatcttcccatgcacaagatgattaatacagatcttagcagaatcttgaaaagcccagagatccaaagagcccttcgagcaccacgcaagaagatccatcgcagagtcctaaagaagaacccactgaaaaacttgagaatcatgttgaagctaaacccatatgcaaagaccatgcgccggaacaccattcttcgccaggccaggaatcacaagctccgggtggataaggcagctgctgcagcagcggcactacaagccaaatcagatgagaaggcggcggttgcaggcaagaagcctgtggtaggtaagaaaggaaagaaggctgctgttggtgttaagaagcagaagaagcctctggtgggaaaaaaggcagcagctaccaagaaaccagcccctgaaaagaagcctgcagagaagaaacctactacagaggagaagaagcctgctgcataaactcttaaatttgattattccataaaggtcaaatcattttggacagcttcttttgaataaagacctgattatacag

SEQ ID NO: 104 - DNA, 317 bp, Artificial
  Synthetic
  misc_feature: SPTAN1/NTRK3
  misc_feature: Druggable kinase DNA chimeras
gccatgcgtcgacgggcccagctagccgattctttccatctgcagcagtttttccgtgattctaatgagctcaagagttgggtcaatgagaagatgaaaactgccacagatgaagcttataaagatccatcccagactaagaagacacaagttctgcttgccatcttgagaggctttccacaggccagaggctgcttcctaacactgacagggcgtgcggtagttaagtcagttttgaagtgagaagcttctggtttctaatatacagtattatcctgatactgcagtcttatgggcaggtgcatgggaaggaaggc

SEQ ID NO: 105 - DNA, 526 bp, Artificial
  Synthetic
  misc_feature: CDK4/IL1R1
  misc_feature: Druggable kinase DNA chimeras
atgtttcttcctctgtttggctttgccattgggcgatttggaaaaaccacttggaaaaagggactttcctgcaaaaccttaaaaactggttaaattacagggcctaggaagtcagtggagccccttgactgacaaagcttaaaaaggaactgaaattgcttctttgaatatggattttaggcggggcgtgggggctggtgacagtaactggtgtttaaatgaaggggaccgtcgctggactggcatgtggtacctgacattcttccaaaaccttgtctttgcaaactggggtccctgtgtaaagtcccctgaccagcggatagccccatgtttctgcttaatgaatttaatcgattctggcattttctcatagtcttggattttctccagctcaagcaggacaactttaattccatcctgaacaagagcattatacatggctatttgctcttcagatgaaccacccagccagctgaagcctgatgtttctctgactaaaatgataatcagacttctgcttttcttt

SEQ ID NO: 106 - DNA, 307 bp, Artificial
  Synthetic
  misc_feature: NTRK3/NTRK3
  misc_feature: Druggable kinase DNA chimeras
acccacgcgtccgcacactcattgtgagccgttccagagagaagaggaagtagggggcaagtatgtcccaaaaaaggatgatttttccaggctcttcctgggcccttagatctgcagttccgacaagctttggctgcaggaggttttacccatgaactggccatcctactaggaccacaagggaccaagggaatcagggacaaaggcccttcctgccagcccatgatcccgggattggctctcttcccctacttccacttattcttgactctgagaacttttggaacccaatggaatcagcatttca

SEQ ID NO: 107 - DNA, 260 bp, Artificial
  Synthetic
  misc_feature: NTRK3/ACTR8
  misc_feature: Druggable kinase DNA chimeras
gtatgtgtagcaggcacttgagtttatatgaagatgttcgcttcagtcaaggagctgtacatcttctttttggtgtcgtcagatgctgaaaagacagttgacatcctccatgcttttttctccacatccataattctaggccaagccaccaacacctcttgcttgagcaagtgcaatagccttctcaaagatttccctccagcccaccctccacactgcagccaggggaaccctacgaaacacaaatgtaggcatgttgc

SEQ ID NO: 108 - DNA, 438 bp, Artificial
  Synthetic
  misc_feature: BRAF/SPAG5-AS1
  misc_feature: Druggable kinase DNA chimeras
agacttctccatgcagacaatctttattatagcagtattcgcatattcacatcaagtacatagaactttttttgccttttatataatacagtttttaaataactttacacagaaataaatttcttcaatctgaatttcagctatctttttttattctccatgctttctatccaaactgaacaatattttctgttatacaaatttacatgagaaaaactccaaagtacaaatgaagggacctgagcaggaaagagaaccaaagtattattattgctcaagaaaggaaacatggcagagaaacaagcagaagcagctctggctgacagcctgagggtaaagagcactggagaagtaaagaaaaggaaaaggtcaccaccctgatctgacatgttgaattgagaggtaggttgtatgatagggaaggaatagggccagctg

SEQ ID NO: 109 - DNA, 476 bp, Artificial
  Synthetic
  misc_feature: MALRD1/ATM
  misc_feature: Druggable kinase DNA chimeras
tatgccttgaggtatagagtaagcaagattttgatttttgctaacgataaatattcatcggtgggcgcagactaggcctatctagggatgtcaggcttggatagcagctaaacttctgccttcaaggattcacattatcatacaaatgtttgcagtgagaaagggtctgatagagagaatataatattacaggaacagatatttcatttggggccagggccaaccattcaagaatctcaaacaatattctgtacctctcacggggttagctagtccaagatctgggatcctaatcacctctctttctttttttgtagagacagggttgccattgtattccagccttggcgacagagcaagactctgcctcaaaaaaaaaaaaaaaaaaggttttggcaagctggaactctttctgccaatgactaagatagaaaactgcccaggacaaattaggagtagttagattttgaaaatat

SEQ ID NO: 110 - DNA, 337 bp, Artificial
  Synthetic
  misc_feature: CDK4/DNASE1
  misc_feature: Druggable kinase DNA chimeras
atgtttcttcctctgtttggctttgcacttgtgcgatttggaaaaaccacttggaagaagggactttcctgcaaaaccttaaagactggttaaattacagggcctaggaagtcagtggagccccttgactgacaaagcttagaaaggaactgaaattgcttctttgaatatggattttaggcggggcgtggtggctcacgcctataataagtttctcacagacacaaggtctccaagtcctgagcccaagcaatcttcccgcaaatgctgggattacaagtgtgagccaccgcaccccgccactcatttcttggaatctttgactttccattcgtat

SEQ ID NO: 111 - DNA, 503 bp, Artificial
  Synthetic
  misc_feature: CDK4/AC007556.1
  misc_feature: Druggable kinase DNA chimeras
atgtttcttcctctgtttggctttgcacttgtgcgatttggaaaaaccacttggaagaagggactttcctgcaaaaccttaaagactggttaaattacagggcctaggaagtcagtggagccccttgactgacaaagcttagaaaggaactgaaattgcttctttgaatatggattttaggcggggcgtggtggctcacgctcataatcctttttaataatacagcatggtggttctgagcatcggctccaacatcaagctgcctgggcttgaaccctggcccacactcactagcagtgtaaccttaggaagttgcttgtctctctgtgattttacttctccatctctggttcataattcaggcatcaacattaattgggtggcattatactcaagttacggttgccatacctaggagaaaacagacccagaacacagtaagtaaatattttatctacatacggggatagatggtttttgcccagaacttaaactatgtattc

SEQ ID NO: 112 - DNA, 288 bp, Artificial
  Synthetic
  misc_feature: CNOT6L/AKT3
  misc_feature: Druggable kinase DNA chimeras
ggaggctctagtttggtgcctagtactagttatttttgttagaacaatctctcaaaatttagataattttccagttgtatgtctgtcacttttaactctaaagcgtaagaatcatggtaaccctctcctccccccgccgtccccgcggctccatcctccgccgccgcccgagcagctgcggggccgccaccgccgccgccgccgttgcaggctgagtcatcactagagagtaggaagggcagcagcagcagagaatccaaaccctaaagctgatatcacgaagtacca

SEQ ID NO: 113 - DNA, 503 bp, Artificial
  Synthetic
  misc_feature: MAP2K1/PDK2
  misc_feature: Druggable kinase DNA chimeras
cagctggaaggggtccccaggagctctgacacagggttttccaactgcccaacatcacggggttggggagcagagcatccagagggctccgtgtattgttttgtagtatagggtctaacttctagaaagtcgggaataggcttctgagagttttggagtcaacaggcctgaatgcagaatgatcaaaagaagatggctattcctctcagagtggtgctggttttcggggccagcagtgcagtgaaaagcccaatttcccaatcctattttgaatatttctgcccccacaagcctagtccatcctgcctgagctccagcccccagcccccactgtgcccagacatgtgtgctcagggtggctttctccctaggaccttctgtgtatatagttagttttataaccctgaatgcccccacccttcccctaagcacacaggggttaaagctgtgtgtccctcccagtggctgtggcagtgacagtgacacccacacccacagtaaag

SEQ ID NO: 114 - DNA, 370 bp, Artificial
  Synthetic
  misc_feature: RAF1/MSS51
  misc_feature: Druggable kinase DNA chimeras
attctggttcctgtatacagtatatctgttcagtatcttaaaaattacaactgttttcatgattttgattgaagatttttttaactcagcccacccacttatggaagtaaagcagaaagggtctcaaagcaactcagaagcctcaggtgcatgatttaaaactcaacatatttatttaaagcagcatctgtcaggcccaaagctcacaacctccttttgggcattaaatttggcatgagccaccatgcccagccaatgtaactggtttctaagagtttagccacagtagcttctagagataggaaattagatttagtgggtgggaagaggccctgatgctggtctttccagagcttatagttgctaacac